SEQUENCE LISTING (10 sequences)

SEQ ID NO: 1 - DNA, 1409 bp, Bos taurus
  source 1..1409: /mol_type=“unassigned DNA” /note=“Bovine MLB nucleotide sequence” /organism=“Bos taurus”
gccctggtgaggatcatgtcgctgtttacatcacttccttttcttctcctgactgcggtgacagcatcttgtgcagacacagaaacagagaactgtgagaacatccggaagacctgccccgtgattgcctgtggtcctccgggcatcaatggcatcccaggcaaagatgggcgtgatggtgccaagggagaaaagggagaaccaggtcaaggactcagaggctcgcagggcccccctggaaagatggggcctcaaggaacgccagggatccctgggataccaggaccaataggccaaaaaggagaccctggagaaaatatgggtgactatattcgcctggctacttcagaaagagcaactctacaatctgaattgaaccagatcaaaaactggctaatcttctctctgggcaaaagagttgggaagaaggcattttttaccaatggtaaaaagatgccttttaatgaagtgaagactctgtgtgcacagttccagggccgtgtggccacccctatgaatgctgaagaaaacagggccctcaaggatttagtcactgaagaggccttcctgggcatcacagatcaggagactgaaggcaaatttgtggatctgacaggaaagggggtgacctaccaaaactggaatgatggcgagcctaacaacgcttctcctggggagcactgtgtgacacttctgtcggacggcacatggaatgacatcgcttgttccgcctcctttttgaccgtctgtgaattctctctctgagggagaatgagcctaaagtcctcctgttcctttactcatctcatgggcccacaacctggtttggaggataaatctatgtcaatttcacacacccagtactgagttgctcttttgtggggaaacagagacaataagaatggattgagaatgatgagatgtggaactgaaaagcgtgaagagacttatagtggtgtaagagtttctggttcagacccagtcactaataataatcatttcaggaacaacaaaataatggtagtaatagtagcaacagcagtaatagtgggagtactaataacatattttaaaatgtttactatgagtcagacattacatgtaagattatacattaagtatctaatttaactaggctgttcatttttgtttgagactataaagaaagctgagcactgaagaattgatggttttgaactgtggtgttggagaagactcttaagagtcccttggactgcaaggagatccaaccagtccatcttaaaggagatcagtcctaggtgttcattggaaggactgaagttgaagctgaaactccaatactttggccacctgatgcaaagagctgactcatttgaaaagaccctgaagctgggaaagattgagggcaggagaaaaagccggaattc

SEQ ID NO: 2 - protein, 249 aa, Bos taurus
  Bovine MLB precursor amino acid sequence
MSLFTSLPFLLLTAVTASCADTETENCENIRKTCPVIACGPPGINGIPGKDGRDGAKGEKGEPGQGLRGSQGPPGKMGPQGTPGIPGIPGPIGQKGDPGENMGDYIRLATSERATLQSELNQIKNWLIFSLGKRVGKKAFFTNGKKMPFNEVKTLCAQFQGRVATPMNAEENRALKDLVTEEAFLGITDQETEGKFVDLTGKGVTYQNWNDGEPNNASPGEHCVTLLSDGTWNDIACSASFLTVCEFSL

SEQ ID NO: 3 - DNA, 1229 bp, Bos taurus
  source 1..1229: /mol_type=“unassigned DNA” /note=“Bovine (bo) MBL CRD pFUSE C4 pFUSE F nucleotide sequence” /organism=“Bos taurus”
tttttttctgttctgcggcgttacagatccagctgtgaccggcgcctacctgagatcaccggcgaaggagggccaccatgtacaggatgcaactcctgtcttgcattgcactaagtcttgcacttgtcacgaattcgatatcggccatggttagatcttggctaatcttctctctgggcaaaagagttgggaagaaggcattttttaccaatggtaaaaagatgccttttaatgaagtgaagactctgtgtgcacagttccagggccgtgtggccacccctatgaatgctgaagaaaacagggccctcaaggatttagtcactgaagaggccttcctgggcatcacagatcaggagactgaaggcaaatttgtggatctgacaggaaagggggtgacctaccaaaactggaatgatggcgagcctaacaacgcttctcctggggagcactgtgtgacacttctgtcggacggcacatggaatgacatcgcttgttccgcctcctttttgaccgtctgtgaattctctctcttaagatctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccggggaggagatgaccaagaaccaggtcagcctgacctgcctggtcaaaggcttctatcccagcgacatcgccgtgggagtggggaggagcatggcagcggagacactacagaccacgcctcccgtgctggactcgacgctctctctctacagcagctcacgtgacagagcagtgcagcaagggacgtcttctcatgctcgtggatgcacgagtctgcaacacttacaccaagctactgttctcggtatgagtgctgactgcacggactgggtaggaaattacccctg

SEQ ID NO: 4 - protein, 127 aa, Mus musculus
  Murine Dectin 1 amino acid sequence
QSCLPNWIMHGKSCYLFSFSGNSWYGSKRHCSQLGAHLLKIDNSKEFEFIESQTSSHRINAFWIGLSRNQSEGPWFWEDGSAFFPNSFQVRNAVPQESLLHNCVWIHGSEVYNQICNTSSYSICEKE

SEQ ID NO: 5 - protein, 127 aa, Homo sapiens
  Human Dectin 1 amino acid sequence
SPCPPNWIIYEKSCYLFSMSLNSWDGSKRQCWQLGSNLLKIDSSNELGFIVKQVSSQPDNSFWIGLSRPQTEVPWLWEDGSTFSSNLFQIRTTATQENPSPNCVWIHVSVIYDQLCSVPSYSICEKK

SEQ ID NO: 6 - protein, 127 aa, Bos taurus
  Bovine Dectin 1 amino acid sequence
SSCPPNWITHEDSCYLFSTLLDSWDGSKRQCFQLGSHLLKIDSSKELEFISRQVSSQPDHSFWIGLSRRQTEEPWLWEDGSTLLSNLFQIRSTVTEKDSSHNCAWIHVSDIYDQLCSVHSYSICEKK

SEQ ID NO: 7 - DNA, 1164 bp, Artificial Sequence
  source 1..1164: /mol_type=“unassigned DNA” /note=“boDect-1 CRD fusion protein nucleotide sequence” /organism=“Artificial Sequence”
atgtacaggatgcaactcctgtcttgcattgcactaagtcttgcacttgtcacgaattcgatatcggccatggttagatctagctcttgtccccctaactggatcacacatgaggatagctgttatctatttagcacactattagattcctgggatggaagtaaaagacaatgctttcaactgggctcccatctcctgaagatagacagctcaaaagagttggagtttatatcaaggcaagtgtcttcccagcctgatcattcattttggatagggctttctcgccgtcagacagaagaaccatggctctgggaggatggctccaccttgttgtctaacctgttccaaatcagaagtacagttaccgaaaaagactcatctcacaactgtgcatggatccatgtgtcagacatttacgaccaactttgtagtgtgcattcatacagtatttgtgagaagaagttgtcagtattaagatctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaatga

SEQ ID NO: 8 - DNA, 533 bp, Artificial Sequence
  source 1..533: /mol_type=“unassigned DNA” /note=“boMBL CRD pFUSE C4 pFUSE R rev nucleotide sequence” /organism=“Artificial Sequence”
tcttgcattgcactaagtcttgcacttgtcacgaattcgatatcggccatggttagatcttggctaatcttctctctgggcaaaagagttgggaagaaggcattttttaccaatggtaaaaagatgccttttaatgaagtgaagactctgtgtgcacagttccagggccgtgtggccacccctatgaatgctgaagaaaacagggccctcaaggatttagtcactgaagaggccttcctgggcatcacagatcaggagactgaaggcaaatttgtggatctgacaggaaagggggtgacctaccaaaactggaatgatggcgagcctaacaacgcttctcctggggagcactgtgtgacacttctgtcggacggcacatggaatgacatcgcttgttccgcctcctttttgaccgtctgtgaattctctctcttaagatctgacaaaactcacacatgcccaccgtgcccagcacctgaactcctggggggaccgtcagtcttcctcttccccccaaacccaagacaccctca

SEQ ID NO: 9 - DNA, 540 bp, Artificial Sequence
  source 1..540: /mol_type=“unassigned DNA” /note=“boMBL CRD pFUSE C4 pFUSE F nucleotide sequence” /organism=“Artificial Sequence”
tcttgcattgcactaagtcttgcacttgtcacgaattcgatatcggccatggttagatcttggctaatcttctctctgggcaaaagagttgggaagaaggcattttttaccaatggtaaaaagatgccttttaatgaagtgaagactctgtgtgcacagttccagggccgtgtggccacccctatgaatgctgaagaaaacagggccctcaaggatttagtcactgaagaggccttcctgggcatcacagatcaggagactgaaggcaaatttgtggatctgacaggaaagggggtgacctaccaaaactggaatgatggcgagcctaacaacgcttctcctggggagcactgtgtgacacttctgtcggacggcacatggaatgacatcgcttgttccgcctcctttttgaccgtctgtgaattctctctcttaagatctgacaaaactcacacatgcccaccgtgcccagcacctgaactcctggggggaccgtcagtcttcctcttccccccaaaccccaaagacaccctcatgatc

SEQ ID NO: 10 - DNA, 375 bp, Bos taurus
  source 1..375: /mol_type=“unassigned DNA” /note=“NM_174107 CRD nucleotide sequence” /organism=“Bos taurus”
tggctaatcttctctctgggcaaaagagttgggaagaaggcattttttaccaatggtaaaaagatgccttttaatgaagtgaagactctgtgtgcacagttccagggccgtgtggccacccctatgaatgctgaagaaaacagggccctcaaggatttagtcactgaagaggccttcctgggcatcacagatcaggagactgaaggcaaatttgtggatctgacaggaaagggggtgacctaccaaaactggaatgatggcgagcctaacaacgcttctcctggggagcactgtgtgacacttctgtcggacggcacatggaatgacatcgcttgttccgcctcctttttgaccgtctgtgaattctctctctga

(19) United States
(12) Patent Application Publication
(10) Pub. No.: US 2016/0280751 A1
(43) Pub. Date: Sep. 29, 2016

(54) COMPOUNDS AND METHODS FOR TREATMENT OR PREVENTION OF A PATHOGEN INFECTION

(71) Applicant: THE ROYAL VETERINARY COLLEGE, London (GB)

(72) Inventor: Dirk Werling, Hatfield (GB)

(21) Appl. No.: 14/903,739

(22) PCT Filed: Jul. 10, 2014
(86) PCT No.: PCT/GB2014/052113

§ 371 Date: Mar. 7, 2016

(30) Foreign Application Priority Data
Jul. 11, 2013 (GB) 1312444.1

(51) Int. Cl. C07K 14/47; A61K 38/17; A61K 45/06

(52) U.S. Cl. CPC C07K 14/4726; A61K 45/06; A61K 38/178; C07K 2319/30

(57) ABSTRACT

A compound comprising a C-type lectin Carbohydrate Recognition Domain (CRD) and an immunoglobulin Fc domain for use in treating or preventing a pathogen infection in a non-human, non-murine subject. Preferably the non-human, non-murine subject is not considered to be an immunocompromised or immunosuppressed subject. The non-human, non-murine subject may be a ruminant; a livestock; companion or racing animal. The CRD, immunoglobulin Fc domain and the non-human, non-murine subject may be bovine. The pathogen infection typically comprises infection by a bacterium, optionally Staphylococcus aureus or Streptococcus Uberis or Streptococcus Agalactiae/dysgalactiae. The subject (for example bovine subject) may have mastitis, subclinical mastitis, acute mastitis, chronic mastitis, high somatic cell count (high SSC), metritis or endometritis. The non-human, non-murine subject may typically be further administered an antibiotic or antifungal agent. The CRD may be from bovine Mannose binding Lectin (MBL) and the immunoglobulin Fc domain may be an IgG1 Fc domain.

### TECHNICAL FIELD

The present invention relates to treatment and prevention of a pathogen infection in non-human animals, for example treatment and prevention of a bacterial infection in livestock such as cattle.

### BACKGROUND OF THE INVENTION

The inflammatory response associated with a pathogen infection, for example a bacterial or viral infection, does not only affect the well-being of an animal, but also decreases production parameters in food-producing animals. Treatment of bacterial infections with new antibiotics becomes more and more problematic due to the increase in multi-drug resistant bacteria as well as the development costs. There is also public pressure to reduce the use of antibiotics in food production to reduce antibiotic residues. We consider that the present invention provides, for example, an approach to tackle some of the most important endemic diseases of high economic importance to the cattle industry in the UK and elsewhere, for example mastitis. Other conditions in which the invention is considered to be useful include metritis/endometritis.

Mastitis, inflammation of the mammary gland, can be caused by a wide range of organisms, including gram-positive bacteria. Within these, Staphylococcus (St.) aureus and Streptococcus (S.) uberis are among the most common etiological agents of bacterial mastitis, and is possibly the most studied mastitis pathogen in dairy cattle. Both pathogens express several factors that compromise the effectiveness of neutrophils and macrophages, immune cell subsets part of the first line of defence against infection in the udder, thus evading destruction. In addition, both pathogens are hard to treat by antibiotics, often resulting in the development of chronic mastitis, leading to increased pain in the animal, reduced production of milk and thus reduced income for the farmer, often resulting in the early euthanisation of the animal due to economic reasons.

Antibiotic treatment of Mastitis caused by Gram-positive bacteria is a long-lasting treatment, often resulting in the development of subclinical Mastitis. The reasons for this are not fully understood, but it seems that the bacteria have developed multiple strategies of “hinding” in the mammary gland. With development costs for new antibiotics increasing, the occurrence of multi-drug resistant bacterial strains in the dairy industry, and the unavailability of a vaccine against Mastitis-causing by Gram-positive bacteria, new intervention strategies need to be identified.

Over the last few years, new groups of innate immune receptors have been identified, expressing carbohydrate-recognition domains (CRD) which recognise a wide variety of pathogens, including, but not limited to, bacteria, viruses and helminths, via specific sugar moieties. Interestingly, these sugar moieties are often shared between pathogens, increasing the spectrum of pathogens recognised by the CRDs.

Polymorph-nucleated neutrophils (PMNs) are well known for their ability in innate immunity to instantly kill pathogens when they invade tissues. However, evidence indicates that PMNs can also directly play a role in adaptive immunity by directly instructing DC and T cells. Upon inflammation, PMNs can travel from the site of infection to the nearest lymph node, where they undergo apoptosis and are taken up by DCs. As a consequence, DCs can present PMN-derived antigens to T cells. In addition, PMNs have been demonstrated to acquire antigen-presenting functions themselves and can directly transfer antigens to DCs. Interestingly, both PMNs and DC express a variety of receptors for antigen-uptake on their surface, including receptors for the recognition of the Fc-part (for example IgG1, IgG2a, but also others) of antibodies (CD16, CD32, CD64, respectively). These receptors are critical surface receptors for facilitating phagocytic movement of antibody-opsonized particles, and ingestion through pathways affecting cytoskeletal reorganization. In addition to these, macrophages and DC (and to a certain degree neutrophils) express C-type lectin receptors (CLRs) that are involved in the recognition and capture of many glycoproteins of pathogens. These CLRs serve as antigen receptors allowing internalization and antigen presentation, but also function as adhesion and/or signaling molecules. The expression of CLRs is very sensitive to maturation stimuli, leading to down-regulation as DCs mature. Membrane-bound CLRs such as DC-SIGN and Dectin-1, as well as secreted CLRs, such as mannose-binding lectin recognize high-mannose/fructose-containing structures expressed on bacteria such as mycobacteria, Gram-positive bacteria, for example staphylococci, streptococci and Listeria, Gram-negative bacteria such as Salmonella and E. coli spp., as well as fungi, such as Candida and Pneumocyctis spp. It was recently demonstrated that glycan modification of antigen can strongly enhance MHC class I responses and the induction of antigen-specific cytotoxic T-lymphocytes, indicating that glycosylated antigen targets

CLRs to enhance antigen-specific T-cell responses. Moreover, these CLRs induce signaling processes in DCs and specific cytokine responses in combination with Toll-like receptor triggering. This implies that specific CLR-targeted antigens can regulate T-cell polarization.

Mattila et al (2008) Antimicrobila Agents and Chemother 52(3), 1171-1172 and Rapaka et al (2007) J Immunol 168, 3702-3712 describe a chimaeric molecule comprising murine Dectin-1 extracellular domain and the Fc portion of murine IgG1 for treating opportunistic fungal infections in immunocompromised subjects. Yabe et al (2010) FEBS J 227(19) 4010-4026 and Hsu et al (2009) J Biol Chem 284(50) 34479-34489 describe the use of similar constructs as tools in investigating binding properties of CRD domains.

### DISCLOSURE OF INVENTION

The present invention provides recombinant polypeptides comprising carbohydrate recognition domains (CRD) of C-type lectins, which may be C-type lectin receptors (CLRs), for use as “artificial” opsonins to, for example, enhance bacterial phagocytosis by cells of the innate immune system, for example polymorph-nucleated neutrophils (PMNs), macrophages (M) and dendritic cells (DC). This is considered to aid in resolution of infection and also in prevention or resolution of subsequent pathogen challenges. The inventors consider that administration of the artificial opsonin leads to faster clearance of pathogens, for example bacteria, and stimulation of the adaptive immune response, as well as reducing the length of antibiotic (for example) treatment necessary. This not only, for example, decreases the risk for antibiotic residues in the milk, but also improves the welfare of the animal faster. Further, since the artificial opsonin is based on naturally occurring structures within the host, the artificial opsonins would not induce an immune response against them, and thus can be used repeatedly.

The inventors recently described the presence of Dectin-1 and DC-SIGN in the bovine system, and have shown that these bind several bacteria. See, for example, Willcocks et al (2006) Veterinary Immunology and Immunopathology. Volume 113, Issues 1-2, 15 Pages 234-242 Identification and gene expression of the bovine C-type lectin Dectin-1; Yamakawa Y et al (2008) J Leukoc Biol. 2008 June; 83(6):1396-403. doi: 10.1189/jlb.0807523. Epub 2008 Mar. 3. Identification and functional characterization of a bovine orthologue to DC-SIGN. The inventors consider that soluble chimeric proteins consisting of the CRD of a CLR and the Fc-part of an IgG molecule enhance phagocytosis of bacteria by PMNs, leading to stimulation of DC and T cells, for example.

### Definitions

As is apparent herein and in accordance with normal usage, the singular form “a”, “an” and “the” include plural references unless the context clearly dictates otherwise. For example, reference to “a pathogen” includes a plurality of such pathogens. As another example, reference to “a C-type lectin” includes a plurality of such C-type lectins.

As is apparent herein and in accordance with normal usage, “a C-type lectin CRD” is intended to mean a carbohydrate recognition domain from any C-type lectin. For example, reference to “a C-type lectin CRD” includes, but is not limited to, a CRD from Mannose binding Lectin, bovine Dectin-1 or DC-SIGN.

As is apparent herein and in accordance with normal usage, the term “for use in treating or preventing a pathogen infection” and the like is intended to mean for therapeutic use against any pathogen infection, such as including, but not limited to, bacterial, mycobacterial, viral, and fungal infections, as well as combinations of such infections.

As is apparent herein and in accordance with normal usage, the term “comprising” is intended to mean that the compositions and methods include the recited elements, but not excluding others.

A first aspect of the invention provides a compound comprising a C-type lectin Carbohydrate Recognition Domain (CRD) and an immunoglobulin Fc domain, or a polynucleotide encoding a polypeptide comprising a C-type lectin Carbohydrate Recognition Domain (CRD) and an immunoglobulin Fc domain, for use in treating or preventing a pathogen infection in a non-human, non-murine subject.

A second aspect of the invention provides the use of a compound comprising a C-type lectin Carbohydrate Recognition Domain (CRD) and an immunoglobulin Fc domain, or a polynucleotide encoding a polypeptide comprising a C-type lectin Carbohydrate

Recognition Domain (CRD) and an immunoglobulin Fc domain, in the manufacture of a medicament for treating or preventing a pathogen infection in a non-human, non-murine subject.

A third aspect of the invention provides a method for treating or preventing a pathogen infection in a non-human, non-murine subject, the method comprising the step of administering to the subject a compound comprising a C-type lectin Carbohydrate Recognition Domain (CRD) and an immunoglobulin Fc domain, or a polynucleotide encoding a polypeptide comprising a C-type lectin Carbohydrate Recognition Domain (CRD) and an immunoglobulin Fc domain.

The following features relate to each of these three aspects of the invention.

In one embodiment, it is considered that the compounds of the present invention are useful in treating animals who may be considered to be normal rather than immunocompromised or immunosuppressed. The inventors have identified that the present invention is useful when the subject is not considered to be an immunocompromised or immunosuppressed subject. Thus, for example, the subject may be a subject who is not receiving treatment intended to significantly functionally to impair the immune response of the subject (for example cyclophosphamide treatment). The subject may be a subject who is not a subject expected to have a functionally impaired immune response from its genetic make-up; or through infection (for example through (immunocompromising) viral infection, for example through HIV or similar infection); or through irradiation.

The inventors consider that the present invention will work alongside and enhance the animal's normal innate and adaptive responses to infection (potentially with significant benefit in improving animal welfare, including reduced suffering of pain and discomfort, agricultural output and efficiency and/or reduction in use of antibiotics) rather than being useful only in compensating for the absence of a normal adaptive response in immunocompromised subjects (for example immunocompromised humans and murine animal models).

The skilled person will readily be able to determine whether an animal is considered to be immunocompromised or immunosuppressed. No complex test is required. The animal will not have been subjected to treatment intended to lead to immunosuppression, or selected as being immunosuppressed. An animal is not considered to be immunocompromised or immunosuppressed merely because the animal has been determined to be infected.

It is further considered that the present invention is useful in treating domesticated animals, for example a livestock; companion or racing animal. For example, the subject may be a ruminant, for example a bovine, sheep or goat. The subject may alternatively be, for example, a pig, horse, poultry (for example chicken, turkey or duck), dog or cat. The subject is not a mouse and typically is not a rodent, for example, a rat, guinea pig or rabbit.

In a particular embodiment, the subject is bovine, for example a dairy cow or beef cow.

In one embodiment, the pathogen infection may comprise infection by a bacterium, typically a Gram-positive bacterium or bacteria. The pathogen infection may comprise any Gram-positive bacteria leading to infection of the respiratory tract, the intestinal tract, the reproductive tract and/or the mammary gland in animals such as cattle, sheep, pigs. Typically the infection may comprise infection by Staphylococcus aureus or Streptococcus Uberis or Streptococcus Agalactiae/dysgalactiae, particularly if the subject is bovine, for example a dairy cow. These bacteria are considered to be the most common etiological agents of bacterial mastitis, as noted above.

The subject (for example bovine subject) may have mastitis, subclinical mastitis, acute mastitis, chronic mastitis, high somatic cell count (high SSC), metritis or endometritis. The subject may be a subject at risk of mastitis, subclinical mastitis, acute mastitis, chronic mastitis, high somatic cell count (high SSC), metritis or endometritis, for example the subject may be part of a herd in which mastitis, subclinical mastitis, acute mastitis, chronic mastitis, high somatic cell count (high SSC), metritis or endometritis is present. These terms are well known to those skilled in the art.

The subject may further be administered one or more further compounds intended to prevent or aid in resolving the infection, for example a known antibiotic or antifungal agent. Many such agents are available and will be well known to those skilled in the art. Antibiotics active against Gram-positive bacteria and non-topical antifungal treatments are considered to be suitable.

See, for example, http://en.wikipedia.org/wiki/Antibacterial and http://en.wikipedia.org/wiki/Anti-fungal_medication and references therein. As noted, antibacterial antibiotics are commonly classified based on their mechanism of action, chemical structure, or spectrum of activity. Most target bacterial functions or growth processes^{[9]} (Calderon C B, Sabundayo B P (2007). Antimicrobial Classifications: Drugs for Bugs. In Schwalbe R, Steele-Moore L, Goodwin A C. Antimicrobial Susceptibility Testing Protocols. CRC Press. Taylor & Frances group. ISBN 978-0-8247-4100-6). Those that target the bacterial cell wall (penicillins and cephalosporins) or the cell membrane (polymixins), or interfere with essential bacterial enzymes (rifamycins, lipiarmycins, quinolones, and sulfonamides) have bactericidal activities. Those that target protein synthesis (macrolides, lincosamides and tetracyclines) are usually bacteriostatic (with the exception of bactericidal aminoglycosides)^{[36]} (Finberg R W, Moellering R C, Tally F P, et al. (November 2004). “The importance of bactericidal drugs: future directions in infectious disease”. Clin. Infect. Dis. 39 (9): 1314-20. doi:10.1086/425009. PMID 15494908). Further categorization is based on their target specificity. “Narrow-spectrum” antibacterial antibiotics target specific types of bacteria, such as Gram-negative or Gram-positive bacteria, whereas broad-spectrum antibiotics affect a wide range of bacteria. Four new classes of antibacterial antibiotics have been brought into clinical use: cyclic lipopeptides (such as daptomycin), glycylcyclines (such as tigecycline), oxazolidinones (such as linezolid) and lipiarmvcins (such as fidaxomicin)^{[37][38]} (Cunha BA. Antibiotic Essentials 2009. Jones & Bartlett Learning, ISBN 978-0-7637-7219-2 p. 180, for example; Srivastava, Aashish; Talaue, Meliza; Liu, Shuang; Degen, David; Ebright, Richard Y; Sineva, Elena; Chakraborty, Anirban; Druzhinin, Sergey Y; Chatterjee, Sujoy; Mukhopadhyay, Jayanta; Ebright, Yon W; Zozula, Alex; Shen, Juan; Sengupta, Sonali; Niedfeldt, Rui Rong; Xin, Cai; Kaneko, Takushi; lrschik, Herbert; Jansen, Rolf; Donadio, Stefano; Connell, Nancy; Ebright, Richard H (2011). “New target for inhibition of bacterial RNA polymerase: ‘switch region’”. Current Opinion in Microbiology 14 (5): 532-43. doi:10.1016/j.mib.2011.07.030. PMC 3196380. PMID 21862392).

Antifungal agents include, for example, polyene antifungals, imidazole, triazole and thiazole antifungals, allylamines, and Echinocandins.

Thus, for example, the invention may be used alongside treatment with a further agent or agents conventionally used in the prevention or treatment of the infection, for example in the case of mastitis, subclinical mastitis, acute mastitis, chronic mastitis, high somatic cell count (high SSC), metritis or endometritis, with an agent or agents conventionally used in the prevention or treatment of mastitis, subclinical mastitis, acute mastitis, chronic mastitis, high somatic cell count (high SSC), metritis or endometritis, as appropriate.

The one or more further agents, for example an antibiotic or antifungal agent, may be administered separately to the subject, for example before or after treatment with the compound of the invention. Alternatively, the one or more further agents may be co-administered to the subject with a CRD-Fc compound of the invention, for example in a co-formulation. Thus, for example, for treatment or prevention of mastitis, subclinical mastitis, acute mastitis, chronic mastitis or high SSC, the CRD-Fc compound and an antibiotic agent may be co-administered by injection into the udder, a delivery route well known for antibiotic treatment of subclinical mastitis, acute mastitis, chronic mastitis or high SSC.

In one embodiment, the pathogen infection may comprise infection by a virus. It is considered that viruses may carry sugar residues, which may be recognised by carbohydrate recognitions domains, in a similar way to those of bacteria, for example. See, for example, Expression of the C-type lectins DC-SIGN or L-SIGN alters host cell susceptibility for the avian coronavirus, infectious bronchitis virus. Zhang Y, Buckles E, Whittaker G R. Vet Microbiol. 2012 Jun. 15; 157(3-4):285-93.doi:10.1016/j.vetmic.2012.01.011. Epub 2012 Jan. 17. The invention may be used alongside treatment with a further agent or agents useful in the prevention or treatment of a viral infection, such as infections by Bovine Viral Diarrhoea Virus, Bovine Respiratory Syncytial virus or Herpesviruses. Many such agents are available and will be well known to those skilled in the art.

In one embodiment, the CRD and immunoglobulin Fc domain are from the same animal species as the subject. Thus, in an example, the CRD and immunoglobulin Fc domain are bovine and the non-human subject is bovine.

The CRD may be from any C-type lectin. In one embodiment, the CRD is from bovine Mannose binding Lectin (MBL), described herein. A compound in which the CRD is from bovine MBL is considered to particularly useful in relation to infection with Gram-positive bacteria, for example Staphylococcus aureus or Streptococcus Uberis or Streptococcus Agalactiae/dysgalactiae. Thus, such a compound may be particularly useful when the subject is bovine, for example a dairy cow; and in relation to treatment or prevention of mastitis, subclinical mastitis, acute mastitis, chronic mastitis, high somatic cell count (high SSC) and metritis/endometritis.

The polypeptide comprising the CRD, for example from bovine MBL, and immunoglobulin Fc domain may form a dimer or higher multiple. A polypeptide comprising the CRD from bovine MBL and immunoglobulin Fc domain is considered to form a dimer and possibly higher multiples.

Other CRD sequences may also be used. For example, as noted below, the CRD sequence may be from bovine DECTIN-1 or DC-SIGN. Typically a CRD sequence is selected that is considered to have adequate binding affinity for sugar moieties expressed by the pathogen of interest. Binding affinity may be measured using techniques well known to those skilled in the art, for example as described herein. As an example, binding affinity may be assessed by the ability to increase the uptake of bacteria in an in-vitro assay. Adequate binding affinity is considered to be present if an increase of at least 20% is achieved, for example, and/or an increase at least 50, 70, 80, 90 or 100% of the increase seen with the bovine MBL CRD.

The term Carbohydrate Recognition Domain (CRD) is well known to the skilled person. This protein domain of approximately 130 amino acids includes a number of invariant cysteine residues. The C-lectin domain is a carbohydrate binding domain that contains a number of invariant cysteine residues, which form disulfide bonds, and that requires calcium ions for binding. The S-lectin domain is a carbohydrate binding domain that contains cysteine residues as free thiols, contains a number of invariant amino acid positions, and does not require divalent cations for binding.

See also, for example, FEBS J. 2011 October; 278(20):3930-41. doi: 10.1111/j.1742-4658.2011.08206.x. Epub 2011 Jul. 1. The carbohydrate recognition domain of collectins. Veldhuizen E J, van Eijik M, Haagsman H P.

Typically the CRD sequence is a wild-type sequence, but a modified variant sequence may be used. However, typically the CDR sequence will be chosen not to elicit an immune response in the intended subject animal, typically of the same species as the CRD sequence was derived from. Thus, the CRD sequences typically will have at least 95%, 96%, 97%, 98%, or 99% sequence identity with the relevant wild-type CRD sequence.

The immunoglobulin Fc domain typically is an IgG1 or IgG2 Fc domain, as well know to those skilled in the art. In one embodiment, the immunoglobulin Fc domain is an IgG1 Fc domain. Typically the Fc domain used is able to bind to PMNs and DC, for example via CD16, CD32, or CD64 receptors. This may be assessed by blocking experiments using antibodies to these receptors, as will be known to those skilled in the art. The Fc domain typically also is able to activate complement. The classical pathway is initiated by binding of complement component C1q to the CH2 domain of an antibody. It is desirable that the Fc domain has the CH2 binding site with the intention that complement activation can occur via the construct. Complement activation is discussed further in Example 2 below. An immunoglobulin “hinge” sequence, as well known to those skilled in the art, may also be present.

In an embodiment, the Fc domain typically is derived from an IgG subclass. It typically is able to activate complement, antibody-dependent cell killing, FcR or complement receptor mediated phagocytosis. It (and/or the construct as a whole, as appropriate) may, for example, be able to stimulate the production of cytokines and/or type I or type II interferons, and/or be able to enhance bacterial/viral elimination.

Whilst not intending to be bound by any one theory, it is envisaged that, in addition or alternative to other potential mechanisms described herein, it is envisaged that the construct polypeptide results in the release of type-I interferon (IFN) from plasmacytoid dendritic cells (pDC). It has been shown that for some viruses, pDC will only be stimulated to release type_I IFN when the virus is taken up in an immune-complexed form (Guzylack-Piriou et al, Europ. J. Immunol. (2006), 36, 1674-16893). As immune-complexed virus, or pathogens in general, will be bound to these cells via the FcR, binding of the CRD-Fc fusion protein to a pathogen via the CR domain is also considered to stimulate the release of type_I IFN from pDC via Fc-FcR interaction. Thus, it is considered that these cells can be stimulated by a complex of the invention to respond to pathogen antigens, such as including, but not limited to, bacterial antigens, with the production of type-I IFN by stimulating a generic mechanism. This is considered to provide a strong anti-viral response (type-I IFN stimulates IFNgamma which activates macrophages and CD8 T cells as well as killer cells), but also to provide a way to stimulate macrophage activation to combat bacterial, mycobacterial or fungal infection, as activated macrophages phagocytose better.

Typically the Fc sequence is a wild-type sequence, but a modified variant sequence may be used. However, typically the Fc sequence will be chosen not to elicit an immune response in the intended subject animal, typically of the same species as the Fc sequence was derived from. Thus, the Fc sequence typically will have at least 95%, 96%_{,} 97%, 98%, or 99% sequence identity with the relevant wild-type Fc sequence.

The compound may comprise a tag sequence, as will be well known to those skilled in the art. The presence of the Fc domain, for example, may make it unnecessary to include a further tag sequence for affinity binding purposes. However, a tag useful in a FRET system may be used. For example, a fluorescent protein tag, for example a Cherry tag may be used. It may be necessary to assess whether a proposed tag interferes to an unacceptable extent with the biological function of the compound and if necessary change or remove the tag.

Numerous further examples of mammalian and non-mammalian CRD and Fc domain polypeptide sequences can be accessed in the sequence databases accessible from the NCBI Medline™ service, as will be well known to the person skilled in the art.

By “variants” of a polypeptide we include insertions, deletions and substitutions, either conservative or non-conservative. In particular we include variants of the polypeptide where such changes do not substantially alter the relevant binding activity (CDR and Fc) or complement activation ability (Fc). The skilled person will readily be able to design and test appropriate variants, based on, for example, comparison of sequences of examples of each polypeptide, for example from different species. The skilled person will readily be able to determine where insertions or deletions can be made; or which residues can appropriately be left unchanged; replaced by a conservative substitution; or replaced by a non-conservative substitution. The variant polypeptides can readily be tested, for example as described in the Examples.

By “conservative substitutions” is intended combinations such as Gly, Ala; Val, Ile, Leu; Asp, Glu; Asn, Gln; Ser, Thr; Lys, Arg; and Phe, Tyr.

The three-letter or one letter amino acid code of the IUPAC-IUB Biochemical Nomenclature Commission is used herein, with the exception of the symbol Zaa, defined above. In particular, Xaa represents any amino acid. It is preferred that at least the amino acids corresponding to the consensus sequences defined herein are L-amino acids.

It is particularly preferred if the polypeptide variant has an amino acid sequence which has at least 90% identity with the amino acid sequence of the relevant wild-type polypeptide, more preferably at least 91%, 92%, 93% or 94%, still more preferably at least 95%, yet still more preferably at least 96%, 97%, 98% or 99% identity with the amino acid sequence of the relevant wild-type polypeptide.

Thus, for example, for a compound comprising a bovine CDR and a bovine Fc domain, it is particularly preferred if the polypeptide variant has an amino acid sequence which has at least 90% identity with the amino acid sequence of the relevant wild-type bovine polypeptide, more preferably at least 91%, 92%, 93% or 94%, still more preferably at least 95%, yet still more preferably at least 96%, 97%, 98% or 99% identity with the amino acid sequence of the relevant wild-type bovine polypeptide.

Typically a CRD variant or Fc domain variant has an amino acid sequence which retains key amino acid motifs characteristic of CRD or Fc domains, respectively, as will be well known to those skilled in the art.

It will be appreciated that the key amino acid motifs characteristic of CRD or Fc domains may be readily identified by a person skilled in the art. For example, as well known to those skilled in the art, it may be desirable to retain glycosylation sites in an Fc domain variant. Glycosylation sites are discussed in, for example, Stadlmann J, Pabst M, Kolarich D, Kunert R, Altmann F. (2008). “Analysis of immunoglobulin glycosylation by LC-ESI-MS of glycopeptides and oligosaccharides”. Proteomics 8 (14): 2858-2871. doi:10.1002/pmic.200700968. PMID 18655055; Stadlmann J, Weber A, Pabst M, Anderle H, Kunert R, Ehrlich H J, Peter Schwarz H, Altmann F. (2009). “A close look at human IgG sialylation and subclass distribution after lectin fractionation”. Proteomics 9 (17): 4143-4153. doi:10.1002/pmic.200800931. PMID 19688751; Peipp M, Lammerts van Bueren J J, Schneider-Merck T, Bleeker W W, Dechant M, Beyer T, Repp R, van Berkel P H, Vink T, van de Winkel J G, Parren P W, Valerius T. (2008). “Antibody fucosylation differentially impacts cytotoxicity mediated by NK and PMN effector cells”. Blood 112 (6): 2390-2399. doi:10.1182/blood-2008-03-144600. PMID 18566325.

The percentage of sequence identity between two polypeptides may be determined using suitable computer programs, for example the GAP program of the University of Wisconsin Genetic Computing Group and it will be appreciated that percent identity is calculated in relation to polypeptides whose sequence has been aligned optimally.

The alignment may alternatively be carried out using the Clustal W program (Thompson et al., 1994). The parameters used may be as follows:

Fast pairwise alignment parameters: K-tuple(word) size; 1, window size; 5, gap penalty; 3, number of top diagonals; 5. Scoring method: x percent.

Multiple alignment parameters: gap open penalty; 10, gap extension penalty; 0.05.

Scoring matrix: BLOSUM.

The alignment may alternatively be carried out using the program T-Coffee, or EMBOSS.

The residue corresponding (equivalent) to, for example, a key amino acid motif characteristic of CRD or Fc domains may be identified by alignment of the sequence of the polypeptide with that of the relevant full-length wild type CRD or Fc domain sequence in such a way as to maximise the match between the sequences. The alignment may be carried out by visual inspection and/or by the use of suitable computer programs, for example the GAP program of the University of Wisconsin Genetic Computing Group, which will also allow the percent identity of the polypeptides to be calculated. The Align program (Pearson (1994) in: Methods in Molecular Biology, Computer Analysis of Sequence Data, Part II (Griffin, AM and Griffin, HG eds) pp 365-389, Humana Press, Clifton). Thus, residues identified in this manner are also “corresponding residues”.

It will be appreciated that in the case of truncated forms of (for example) the MBL CRD, or in forms where simple replacements of amino acids have occurred it is facile to identify the “corresponding residue”.

It is preferred that the domains used in the compound are mammalian, preferably a species useful in agriculture or as a domesticated or companion animal, for example dog, cat, horse, cow), including naturally occurring allelic variants (including splice variants).

The pathogen infection may comprise infection by a mycobacterium, optionally Mycobacterium bovis; or a fungus, such as Candida albicans or any other yeast-subspecies or fungal sub-species such as Pneumocystis. It may be useful, particularly in such cases, for the CRD to be from bovine DECTIN-1 or DC-SIGN or MBL.

In one embodiment, an antifungal or antibiotic compound may be administered to the non-human subject before, during, or after treatment with a compound of the invention. Typical antifungal agents will be well know to those skilled in the art and include Amphotericin B, Nystatin, Clotrimazol, Tolnaftate, Crystal violet. Typical antibiotics will also be well known to those skilled in the art.

A further aspect of the invention provides a compound comprising a non-human, non-murine C-type lectin Carbohydrate Recognition Domain (CRD) and a non-human, non-murine immunoglobulin Fc domain, optionally wherein the CRD and the immunoglobulin Fc domain are bovine, optionally wherein the CRD domain is from bovine Mannose Binding Lectin or bovine Dectin-1 or DC-SIGN. Further preferences for the compound and its components are indicated above.

A further aspect of the invention provides a polynucleotide encoding a compound of the preceding aspect of the invention. A further aspect of the invention provides a polynucleotide vector molecule comprising a polynucleotide of the invention and capable of expressing a compound of the invention. A still further aspect of the invention provides a host cell comprising a polynucleotide or polynucleotide vector molecule of the invention.

Typical prokaryotic vector plasmids are: pUC18, pUC19, pBR322 and pBR329 available from Biorad Laboratories (Richmond, Calif., USA); pTrc99A, pKK223-3, pKK233-3, pDR540 and pRIT5 available from Pharmacia (Piscataway, N.J., USA); pBS vectors, Phagescript vectors, Bluescript vectors, pNH8A, pNH16A, pNH18A, pNH46A available from Stratagene Cloning Systems (La Jolla, Calif. 92037, USA).

A typical mammalian cell vector plasmid is pSVL available from Pharmacia (Piscataway, N.J., USA). This vector uses the SV40 late promoter to drive expression of cloned genes, the highest level of expression being found in T antigen-producing cells, such as COS-1 cells. An example of an inducible mammalian expression vector is pMSG, also available from Pharmacia (Piscataway, N.J., USA). This vector uses the glucocorticoid-inducible promoter of the mouse mammary tumour virus long terminal repeat to drive expression of the cloned gene.

Useful yeast plasmid vectors are pRS403-406 and pRS413-416 and are generally available from Stratagene Cloning Systems (La Jolla, Calif. 92037, USA). Plasmids pRS403, pRS404, pRS405 and pRS406 are Yeast Integrating plasmids (Ylps) and incorporate the yeast selectable markers HIS3, TRP1, LEU2 and URA3. Plasmids pRS413-416 are Yeast Centromere plasmids (YCps).

Methods well known to those skilled in the art, for example using PCR can be used to construct expression vectors containing the coding sequence and, for example appropriate transcriptional or translational controls.

The polynucleotide may be expressed in a suitable host (which may typically be an eukaryotic host) to produce a polypeptide comprising the compound of the invention. Thus, the DNA encoding the polypeptide constituting the compound of the invention may be used in accordance with known techniques, appropriately modified in view of the teachings contained herein, to construct an expression vector, which is then used to transform an appropriate host cell for the expression and production of the polypeptide of the invention.

Such techniques will be well known to those skilled in the art and include those disclosed in U.S. Pat. No. 4,440,859 issued 3 Apr. 1984 to Rutter et al, U.S. Pat. No. 4,530,901 issued 23 Jul. 1985 to Weissman, U.S. Pat. No. 4,582,800 issued 15 Apr. 1986 to Crowl, U.S. Pat. No. 4,677,063 issued 30 Jun. 1987 to Mark et al, U.S. Pat. No. 4,678,751 issued 7 Jul. 1987 to Goeddel, U.S. Pat. No. 4,704,362 issued 3 Nov. 1987 to Itakura et al, U.S. Pat. No. 4,710,463 issued 1 Dec. 1987 to Murray, U.S. Pat. No. 4,757,006 issued 12 Jul. 1988 to Toole, Jr. et al, U.S. Pat. No. 4,766,075 issued 23 Aug. 1988 to Goeddel et al and U.S. Pat. No. 4,810,648 issued 7 Mar. 1989 to Stalker, all of which are incorporated herein by reference.

The DNA encoding the polypeptide constituting the compound of the invention may be joined to a wide variety of other DNA sequences for introduction into an appropriate host. The companion DNA will depend upon the nature of the host, the manner of the introduction of the DNA into the host, and whether episomal maintenance or integration is desired.

Generally, the DNA is inserted into an expression vector, such as a plasmid, in proper orientation and correct reading frame for expression. If necessary, the DNA may be linked to the appropriate transcriptional and translational regulatory control nucleotide sequences recognised by the desired host, although such controls are generally available in the expression vector. Thus, the DNA insert may be operatively linked to an appropriate promoter. Bacterial promoters include the E. coli lacI and lacZ promoters, the T3 and T7 promoters, the gpt promoter, the phage λ PR and PL promoters, the phoA promoter and the trp promoter. Eukaryotic promoters include the CMV immediate early promoter, the HSV thymidine kinase promoter, the early and late SV40 promoters and the promoters of retroviral LTRs. Other suitable promoters will be known to the skilled artisan. The expression constructs will desirably also contain sites for transcription initiation and termination, and in the transcribed region, a ribosome binding site for translation. (Hastings et al, International Patent No. WO 98/16643, published 23 Apr. 1998).

The vector is then introduced into the host through standard techniques. Generally, not all of the hosts will be transformed by the vector. It will, therefore, be necessary to select for transformed host cells. One selection technique involves incorporating into the expression vector a DNA sequence marker, with any necessary control elements, that codes for a selectable trait in the transformed cell, such as antibiotic resistance. These markers include dihydrofolate reductase, G418 or neomycin resistance for eukaryotic cell culture, and tetracyclin, kanamycin or ampicillin resistance genes for culturing in E. coli and other bacteria. Alternatively, the gene for such selectable trait can be on another vector, which is used to co-transform the desired host cell.

Host cells that have been transformed by the recombinant DNA of the invention are then cultured for a sufficient time and under appropriate conditions known to those skilled in the art in view of the teachings disclosed herein to permit the expression of the polypeptide, which can then be recovered.

The polypeptide of the invention can be recovered and purified from recombinant cell cultures by well-known methods including ammonium sulphate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, immunoglobulin columns or high performance liquid chromatography (“HPLC”) is employed for purification.

Many expression systems are known, including bacteria (for example E. coli and Bacillus subtilis), yeasts (for example Saccharomyces cerevisiae), filamentous fungi (for example Aspergillus), plant cells, animal cells and insect cells.

Expression systems include, but are not limited to: microorganisms such as bacteria transformed with recombinant bacteriophage, plasmid or cosmid DNA expression vectors; yeast transformed with yeast expression vectors; insect cell systems transformed with viral expression vectors (eg. baculovirus); plant cell systems transfected with viral or bacterial expression vectors; animal cell systems transfected with adenovirus expression vectors.

Many expression systems are known, including systems employing: bacteria (eg. E. coli and Bacillus subtilis) transformed with, for example, recombinant bacteriophage, plasmid or cosmid DNA expression vectors; yeasts (eg. Saccaromyces cerevisiae) transformed with, for example, yeast expression vectors; insect cell systems transformed with, for example, viral expression vectors (eg. baculovirus); plant cell systems transfected with, for example viral or bacterial expression vectors; animal cell systems transfected with, for example, adenovirus expression vectors.

A typical mammalian cell vector plasmid is pSVL available from Pharmacia (Piscataway, N.J., USA)., Piscataway, N.J., USA This vector uses the SV40 late promoter to drive expression of cloned genes, the highest level of expression being found in T antigen-producing cells, such as COS-1 cells.

An example of an inducible mammalian expression vector is pMSG, also available from Pharmacia (Piscataway, N.J., USA). This vector uses the glucocorticoid-inducible promoter of the mouse mammary tumour virus long terminal repeat to drive expression of the cloned gene.

Useful yeast plasmid vectors are pRS403-406 and pRS413-416 and are generally available from Stratagene Cloning Systems (La Jolla, Calif. 92037, USA), La Jolla, Calif. 92037, USA. Plasmids pRS403, pRS404, pRS405 and pRS406 are Yeast Integrating plasmids (Ylps) and incorporate the yeast selectable markers HIS3, TRP1, LEU2 and URA3. Plasmids pRS413-416 are Yeast Centromere plasmids (YCps).

A further aspect of the invention provides a compound of the invention or a polynucleotide or vector molecule or host cell of the invention for use in medicine.

A further aspect of the invention provides a pharmaceutical formulation comprising a compound, polynucleotide, vector molecule or host cell of the invention. The pharmaceutical formulation may further comprise a further antibiotic or antifungal agent. Characteristics of pharmaceutical formulations will be well known to those skilled in the art. Suitable pharmaceutical formulations may be prepared using known techniques suitable for, for example, the nucleic acid or polypeptide compounds and optional antibiotic or antifungal agents relevant to the present invention.

A further aspect of the invention provides an antibiotic or antifungal agent for use in treating a non-human, non-murine subject, wherein the subject is administered a compound comprising a C-type lectin Carbohydrate Recognition Domain (CRD) and an immunoglobulin Fc domain.

The listing or discussion of an apparently prior-published document in this specification should not necessarily be taken as an acknowledgement that the document is part of the state of the art or is common general knowledge.

Preferences and options for a given aspect, feature or parameter of the invention should, unless the context indicates otherwise, be regarded as having been disclosed in combination with any and all preferences and options for all other aspects, features and parameters of the invention.

The invention is now described in more detail by reference to the following, non-limiting Figures and Examples.

### DESCRIPTION OF THE DRAWINGS

FIG. 1: The CRD of the innate immune receptor Dectin-1 and the Fc fragment of a human IgG1 antibody are cloned to produce the Dectin-1 CRD-Fc fusion protein.

Concentrated supernatants and purified proteins from transfected HEK293 cells were probed to see if the fusion proteins were produced correctly. Bovine Dectin-1 CRD-Fc showed bands for both the Fc Fragment and Dectin-1 at the same size, showing correct expression of the protein.

FIG. 2: Diagram of fusion polypeptide and source of domains

FIGS. 3 and 4: Quantification of the concentrated supernatant using the Agilent Bioanalyzer showed a good yield of protein per transfection.

FIG. 5: Binding specificity of Bovine Dectin-1 was compared with human and murine Dectin-1 using an oligosaccharide microarray. Binding data shows that Bovine Dectin-1 is also highly restricted to the long beta1-3-linked gluco-oligosaccharide chains.

FIG. 6: Effect of concentration of protein on Zymosan uptake. Results formed from 4 repeats

FIG. 7: Percentage uptake of Zymosan beads over time. Representative of 4 repeats

FIG. 8: Complement activation of C3, iC3b, C4 and C5b-9 by solid phase ELISA. HAS negative control and IgG complex positive control. Significance values given are compared to HAS 20 μg.P values *=<0.05 **=<0.01 ***=<0.001. See also FIG. 18 for diagram of assay arrangement.

FIG. 9: Complement activation by various proteins tested using fluid phase assays. Zymosan and IgG complex act as positive controls for complement activation and HAS acts as the negative control for background complement activation.

FIG. 10: Physiological complement activation by Zymosan BioParticles™ analysed by flow cytometry. Complement components bound to Zymosan BioParticles™ were compared with and without treatment with Fc Fragment and bovine Dectin-1 CRD-Fc. No serum and EDTA repeat act as controls. See also FIG. 19 for diagram of assay arrangement.

FIG. 11: sequence analysis relating to bovine MBL: Fc fragment fusion.

FIG. 12: plasmid diagram relating to bovine MBL: Fc fragment fusion.

FIG. 13: sequence confirmation relating to bovine MBL: Fc fragment fusion.

FIG. 14: Bacterial killing. See Example 6

FIG. 15: multiple alignment of Dectin-1 sequences

FIG. 16: plasmid diagram relating to bovine Dectin-1:Fc fragment fusion.

FIG. 17: Western blotting of expressed proteins. Proteins blotted on 4-20% SDS-PAGE gels and subsequently probed for Fc fragment or Dectin-1. This confirms that the protein has been expressed correctly.

FIG. 18: Diagram of complement assay—ELISA plate

FIG. 19: Diagram of complement assay—Zymosan BioParticle™

FIG. 20: Promega ADCC assay was used to measure ADCC. Ready to use effector NK cells were incubated for 6 hours with the fusion protein, or negative control and effector Zymosan Bioparticles™. Raji cells and anti CD20 antibody was used as a positive control.

FIG. 21: The fusion protein enhances uptake in macrophages (M). Macrophages or neutrophils (see FIG. 22) were incubated with fluorescent Zymosan BioParticles™ with the full fusion protein or just the Fc fragment. The effect of concentration or time on the uptake of Zymosan BioParticles™ was measured using flow cytometry. Data was analysed using Flowjo (Treestar In.) and SPSS (IGM) and statistics derived using linear mixed modeling with LSD post-hoc analysis. Data is from four animals, showing the uptake of Zymosan bead. P values *<0.05, ***<0.01, ***<0.001.

FIG. 22: The fusion protein enhances uptake in PMNs. Data is from four animals, showing the uptake of Zymosan bead. P values *<0.05, **<0.01, ***<0.001.

FIG. 23: Macrophages or neutrophils (see FIG. 24) were used for phagocytosis uptake assays. Cells were stimulated with Zymosan alone or with bovine Dectin-1 CRD-Fc, bovine Dectin-1 CRD or Fc Fragment. In addition FcγRII was blocked. Phagocytosis was measured over time and results analysed using flow cytometry and Flowjo. M incubated with either boDectin-1 CRD-Fc, boDectin-1 CRD, Fc Fragment or boDectin-1 CRD-Fc with anti-CD32 antibody and uptake measured over time in percentage and MFI. Data from 3 experiments.

FIG. 24: PMN incubated with either boDectin-1 CRD-Fc, boDectin-1 CRD, Fc Fragment or boDectin-1 CRD-Fc with anti-CD32 antibody and uptake measured over time in percentage and MFI. Data from 3 experiments.

The fusion protein significantly opsonises pathogens in macrophages and neutrophils in a time and concentration dependant manner. Blocking of FcgammaR11 did show a trend for reduction of uptake in macrophages, but was not significant. In neutrophils the opposite is true. Only the full protein opsonised uptake in macrophages. In neutrophils other protein may enhance uptake, but the fusion protein is considered to enhance uptake more

FIG. 25: Macrophages and neutrophils were used for analysis of intracellular ROS (reactive oxygen species) and NOS (nitrous oxide synthase) production after phagocytosis. Cells were stimulated with S. Cerevisiae or Zymosan alone with or without bovine Dectin-1 CRD-Fc. ROS and NOS were measured after 2 hours using flow cytometry and data analysed using Flowjo. Data from 4 animals, showing MFI of ROS or NOS. P values *<0.05, **<0.01, ***<0.001.

FIG. 26: Macrophages were incubated with various treatments in order to assess cytokine production. Supernatants were harvested at 1 hr, 6 hr and 24 hr. Supernatants were analysed for cytokines using an ELISA multiplex. Data from 3 animals, showing production of cytokine. P values *<0.05, **<0.01, ***<0.001.

The fusion protein does not significantly increase ROS or NOS in macrophages. ROS is significantly increased in neutrophils after the addition of the fusion protein. IL12 is significantly reduced compared to Zymosan alone after the addition of the fusion protein. This may mean that the construct induces a different type of immune response. IL-12 is a marker for a cell-mediated, Th1 response. As the amount of IL-12 released is reduced, the response may be skewed towards a Th2 type response.

FIG. 27: Production of Type I-IFN by pDC cells. pDC were generated as described and Type I IFN analysed as described in “Identification of a lineage negative cell population in bovine peripheral blood with the ability to mount a strong type I interferon response” Gibson A, Miah S, Griebel P, Brownlie J, Werling D. Dev Comp Immunol. 2012 February; 36(2):332-41. doi: 10.1016/j.dci.2011.05.002. Epub 2011 May 31. Cells were incubated with the same amount of zymosan and zymosan+ CRD-Fc (CRD from dectin-1) as in the other experiments (for example FIG. 21), and IFN measured. The pDC produce more IFN with the zymosan+ CRD-Fc. This fits to the idea that a complexed pathogen may stimulate type I IFN release from these cells via FcR activation.

FIG. 28: sequence analysis relating to bovine Dectin: human Fc fragment fusion.

### EXAMPLES

### Example 1

### The Use of CRD-Fc Proteins as Artificial Opsonins to Enhance Pathogen Killing

Polymorph-nucleated neutrophils (PMNs) and macrophages (M) are well known for their ability in innate immunity to instantly kill pathogens when they invade tissues. Interestingly, both M and PMNs express a variety of receptors for antigen uptake on their surface, including receptors for the recognition of the Fc part of antibodies. These receptors are critical surface receptors for facilitating phagocytic movement of antibody opsonized particles, and ingestion through pathways affecting cytoskeletal reorganization. In addition to FcR, both cell types express CLRs (C-type lectin receptors) that are involved in the recognition and capture of many glycosylated self antigens and pathogens. These CLRs serve as antigen receptors allowing internalization and antigen presentation, but also function as adhesion and/or signaling molecules. CLRs such as Dectin-1 recognize high mannose containing structures expressed on pathogens such as staphylococci and yeast. Moreover, these CLRs induce signaling processes and specific cytokine responses in combination with TLR (Toll-like receptor) activation. To aid phagocytosis of invading pathogens, fusion proteins of CLRs are considered to provide a novel and exciting approach to enhance pathogen killing, for example by administrating these together with antibiotics. By fusing the carbohydrate recognition domain (CRD) of bovine innate immune receptors/polypeptides to a Fc fragment of either bovine or human IgG1, the inventors prepared an artificial opsonin. This opsonin was able to recognise conserved structures on the surface of pathogens and enhance their uptake by binding to the FcR expressed on phagocytic cells.

The potential of CRDs as “artificial” opsonins can be assessed by:

### 1) Cloning, Sequencing and Expressing CRD Domains of Murine/Human/Bovine Dectin-1/DC-SIGN/MBL as Fc-Tagged Proteins

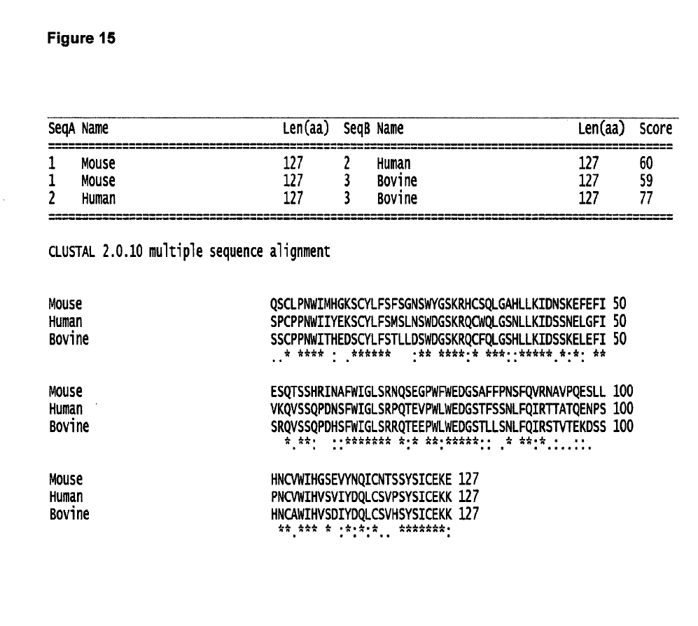

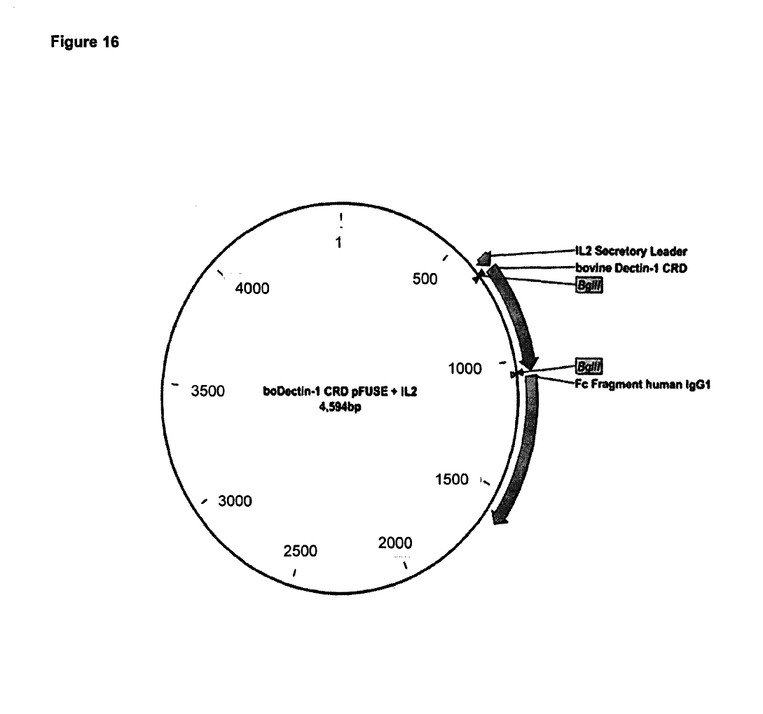

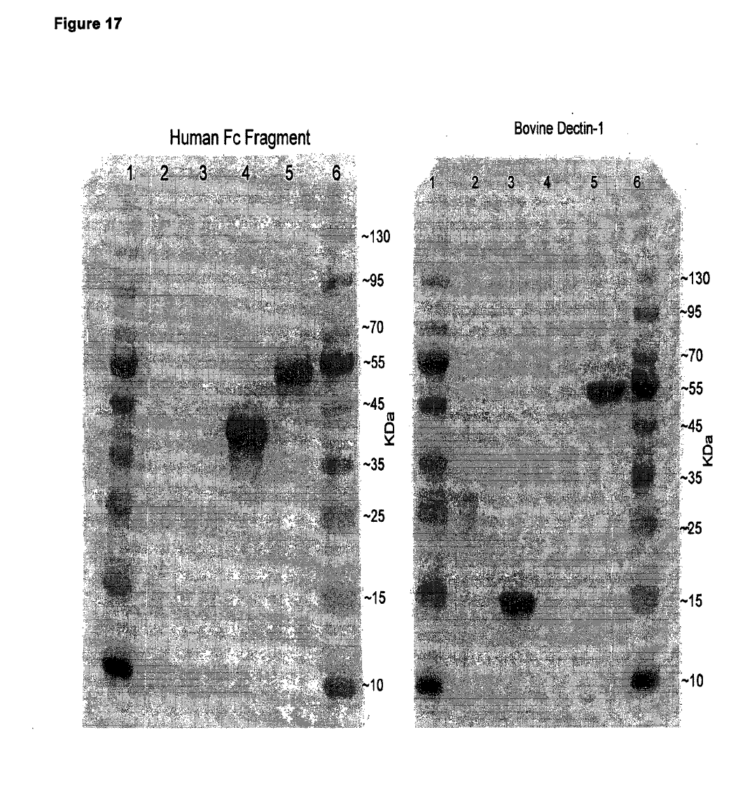

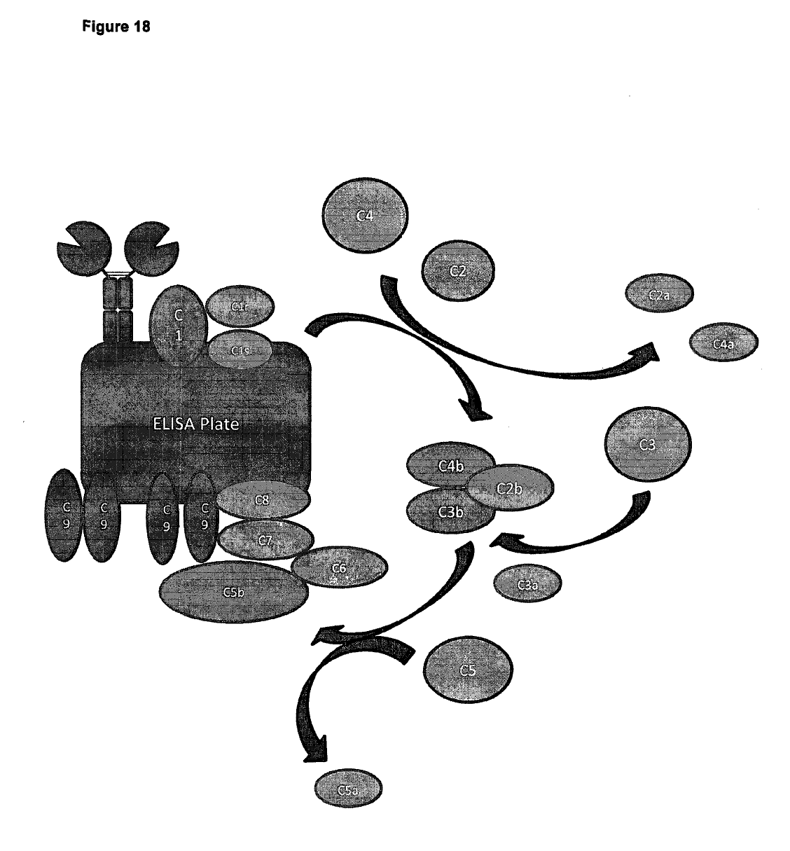

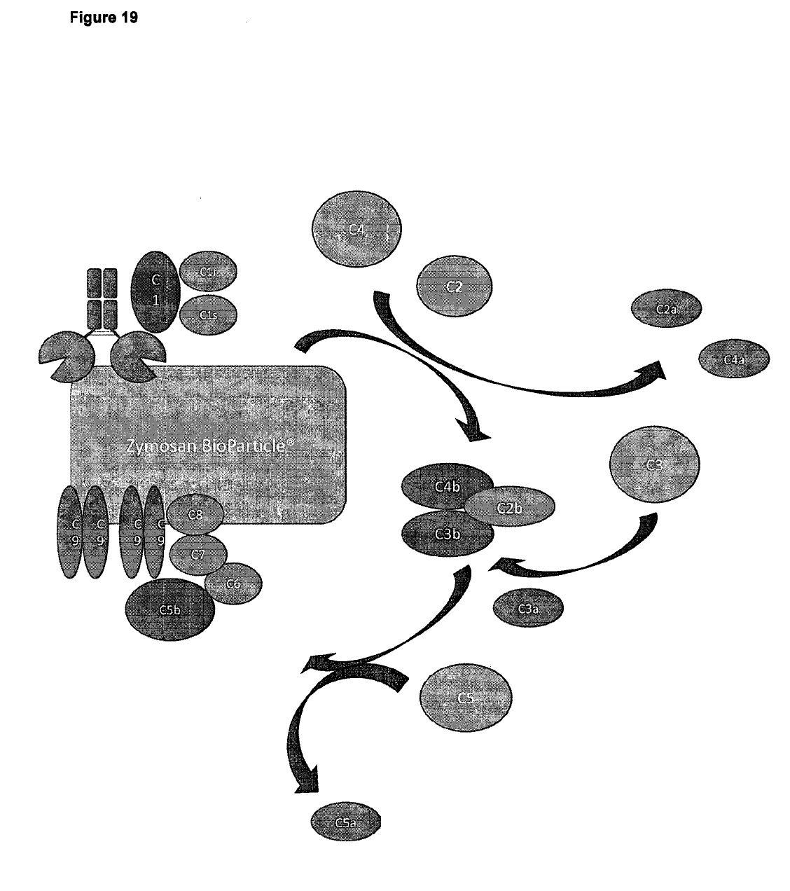

The sequences of bovine dectin-1. DC-SIGN and MBL have been identified and human and murine sequences are also known. An alignment of mouse, human and bovine Dectin-1 is shown in FIG. 15. A plasmid containing the full-length murine Dectin-1 receptor was provided by G. Brown (University of Cape Town, Cape Town, South Africa), and the CRD of this molecule was already cloned into the pSecTag expression vector, containing the IgK leader sequence facilitating protein secretion. The CRD of the remaining molecules may similarly be cloned. For protein generation. HEK293 cells can be transiently transfected with the resulting constructs, and supernatants harvested. If necessary, CRD-Fc proteins can be affinity purified over a Sepharose column using goat-anti-mouse H chain IgG.

### 2) Assessing the Functionality of CRD-Fc Proteins by Assessing Their Ability to Opsonise Bacteria and to Induce Increased Phagozytosis in Different Cell-Types

To assess the functional properties of CRD-FC proteins, several techniques can be used. To assess the binding properties, bacterial organisms (M. bovis. St. aureus and S. uberis) can be fixed to glass slides and incubated with CRD-Fc proteins or control medium, and bound CRD-Fc can be detected by incubating with fluorochrome-conjugated IgG using a confocal laser-scanning microscope. Furthermore, cells can be co-cultured with different concentrations of either bacteria; or be pre-opsonized with either CRD-Fc proteins or controls before incubated with cells. After various time-points, cells can be lysed and viability of bacteria assessed by re-cultivation. To assess cell surface association with either bacteria expressing GFP, bacteria can be incubated with cells, in the presence or absence of laminarin (to block dectin-1, as cross-reacting antibodies are not yet available) and existing antibodies to CD11/18, CD66a (CEACAM1) and Fc RII/III. As control, FITC-zymosan (Molecular Probes) can be used, and association can be analysed using a flow cytometer for relative FITC intensity.

### 3) Assessing Effects of CRD-Fc Protein Opsonised Bacteria on PMN Function

Recognition/uptake of bacteria by PMN/DC is known to induce their maturation as well as the secretion of cytokines/chemokines. Changes in maturation markers, such as CD40 and CD80/86 can be assessed by flow cytometry. Several chemokines have recently been cloned and these can be analysed by either qPCR or functional assays. For example, those chemokines which have been shown to enhance interaction of PMNs with DCs (CXCL8, CXCL1, CCL3 and CCL4) can be analysed. Furthermore, the production of TNF by PMNs can be analysed, as this is known to be involved in apoptosis induction, as well as the induction of IL-12 by DC. Chemokines/cytokines can be assessed after exposure of PMNs to media, bacteria or CRD-Fc protein opsonised bacteria over a time-course.

### 4) Assessing the Ability of PMNs to Transfer Phagocytised Bacteria to DC and to Mount a Subsequent CD4 or CD8 Response.

The above experiments can aid in demonstrating that CRD-Fc increase the phagocytosis and subsequent killing of opsonised bacteria. To demonstrate that this also has an effect on the subsequent T cell response generated, cells can be incubated either alone or after co-cultivation with sorted T cell subsets to assess the effect of CRD-Fc protein opsonised bacteria on the induction of the adaptive immune response.

### Materials and Methods

### Expression of Fusion Proteins

HEK 293 cells were transiently transfected with the bovine Dectin-1 CRD-Fc (human) fusion proteins using Turbofect™ (Fermentas). Briefly, adhered cells were gently washed with warmed Opti-MEM® (Gibco). 10 μg DNA was mixed with Turbofect™ diluted in Opti-MEM®. Cells were incubated with DNA and Turbofect™ for 5 hours, then 7 mls complete media added (RPMI, 20% FCS, 4% Pen/Strep). After 24 hours, the media was removed and cells washed with warm Opti-MEM®. Then, 14 ml of RPMI media was added and cells left for 36 hours before removing the supernatant.

### Concentrating Cell Culture Supernatants

Cell culture supernatants were placed in Amicon 30 kd molecular cut off centrifugation columns (Millipore). Samples were spun at 4000× g for 15 minutes. Flow through was discarded. Concentrated samples were diluted with fresh RPMI and spun as before. Recovered concentrate was stored at −20° C.

### Western Blotting

Protein lysates were boiled at 95° C. with LDS buffer and DTT reducer (Expedeon). Samples were loaded onto precast 4-20% SDS Page gels (Expedeon) and run at 180V for 45 minutes in a rapid SDS reducing buffer (Expedeon). SDS Page gels were transferred onto prepared PVDF membrane at 100V for 75 minutes using a Tris/Glycine buffer (Biorad) with 20% methanol. Membranes were blocked and probed using the SNAP i.d.™ system (Millipore). Membranes were probed with primary antibody for either anti-human IgG1 Fc (Abd Serotec) or anti-Dectin-1 (Antibodies Online). After washing, membranes were probed with either anti-rabbit or anti-mouse HRP secondary antibodies (Amersham). Bands were detected using ECL Plus Kit (Amersham) and Hyperfilm (GE Healthcare), films were processed automatically using an AGFA Curix 60 (AGFA).

### Quantification of Protein

Concentrated supernatants were analysed using P230 Assay Chips on the Bioanalyzer 2100 (Agilent) to quantify the amount of protein present in each sample.

### Macrophage Phagocytosis Assay

Alexaflour® 594 labeled Zymosan BioParticles® were pre-incubated for 1 hr at 37° C. with Fc Fragment, bovine Dectin-1 CRD-Fc or no protein in RPMI. Bovine monocyte derived macrophages were added to each sample and incubated for another 1 hr at 37° C. Cells were washed quickly 3 times and analysed using flow cytometry looking for macrophages which had taken up fluorescent BioParticles®. Assays were performed at time intervals and varying concentrations.

### Expression of CRD-Fc Fusion Proteins

The CRD of the innate immune receptor bovine Dectin-1 and the Fc fragment of a human IgG1 antibody are cloned to produce the Dectin-1 CRD-Fc fusion protein.

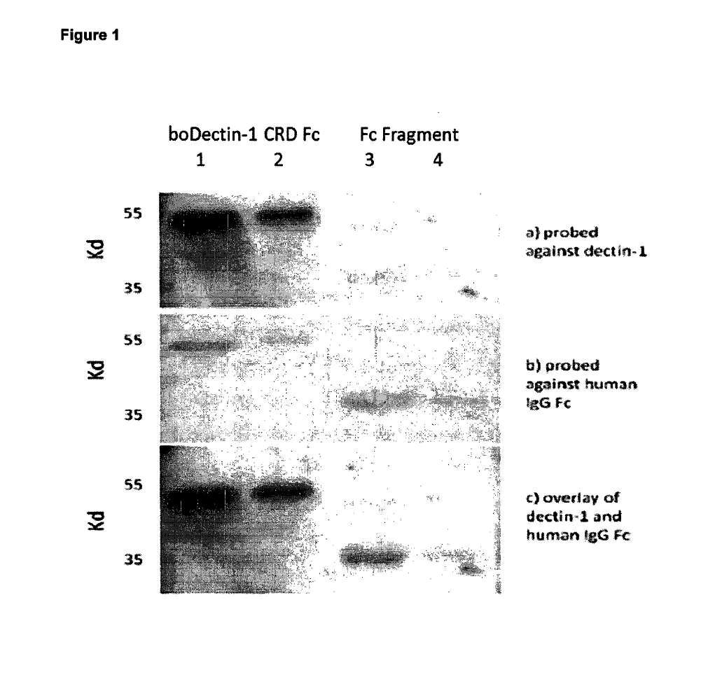

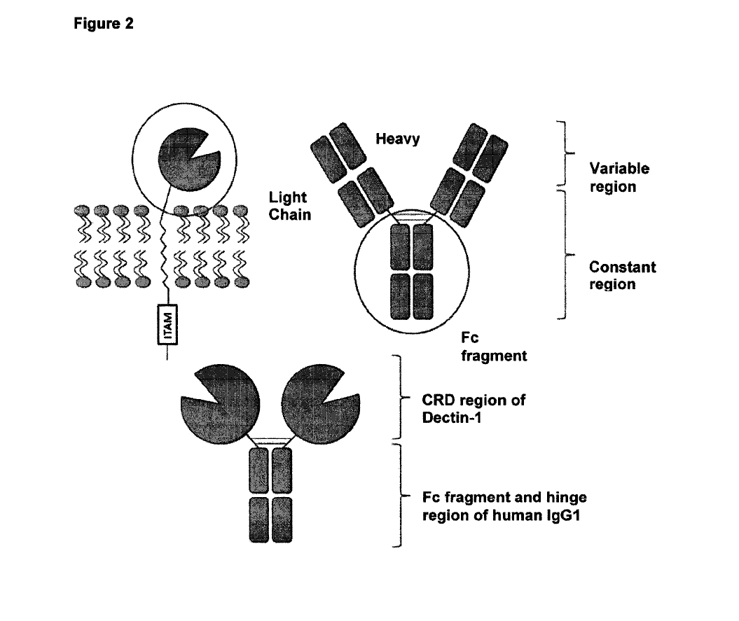

Concentrated supernatants and purified proteins from transfected HEK293 cells were probed to see if the fusion proteins were produced correctly. Bovine Dectin-1 CRD-Fc showed bands for both the Fc Fragment and Dectin-1 at the same size, showing correct expression of the protein. See FIGS. 1 and 2.

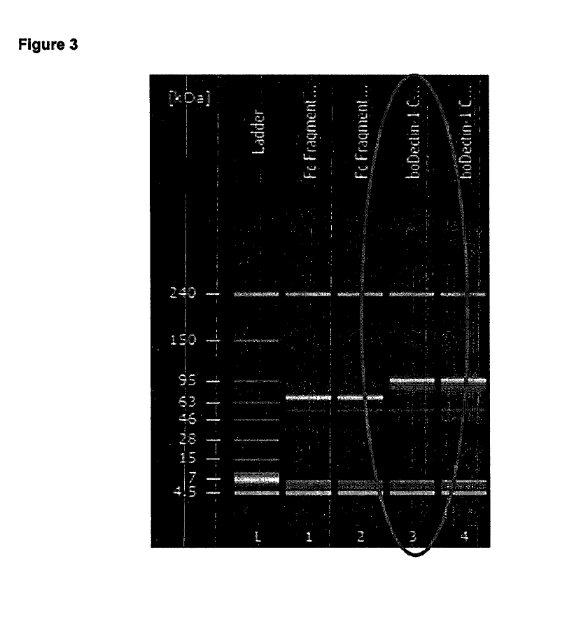

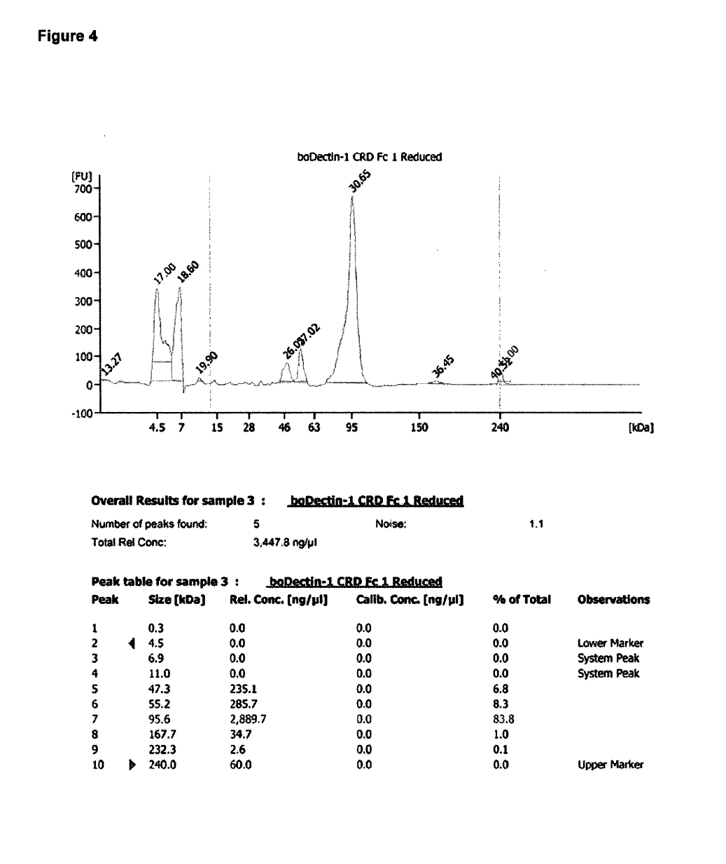

Quantification of the concentrated supernatant using the Agilent Bioanalyzer showed a good yield of protein per transfection. See FIGS. 3 and 4.

### Binding Specificity of Bovine Dectin-1

Binding specificity of Bovine Dectin-1 was compared with human and murine Dectin-1 using an oligosaccharide microarray. Binding data shows that Bovine Dectin-1 is also highly restricted to the long beta1-3-linked gluco-oligosaccharide chains. See FIG. 5.

### Phagocytosis Assays

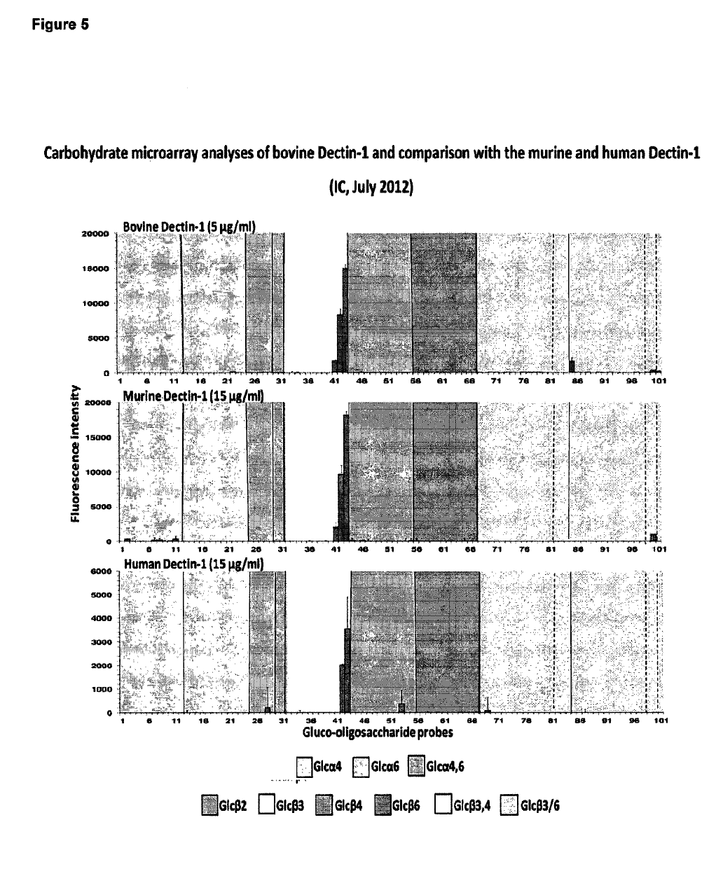

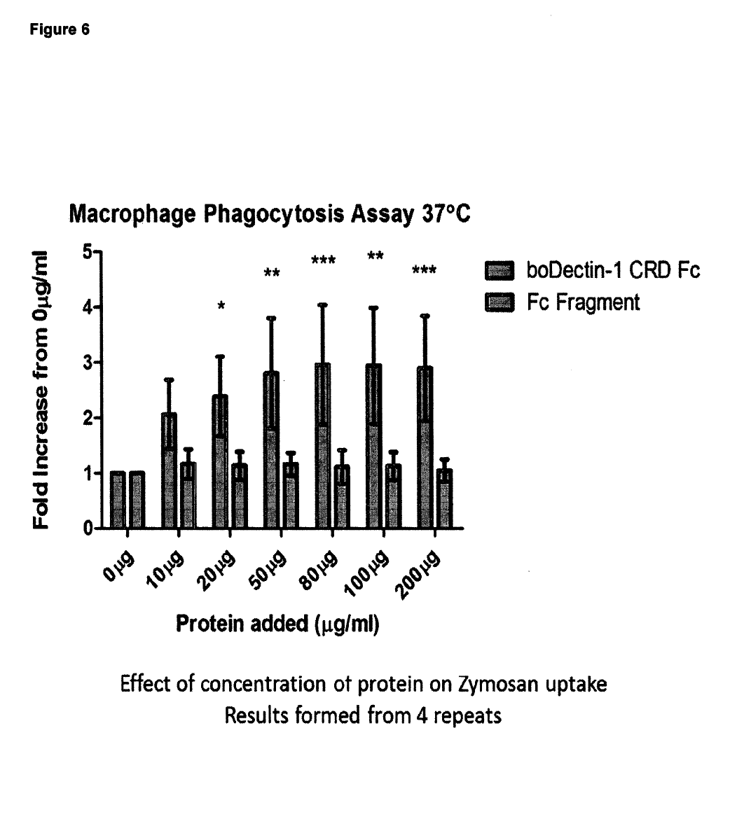

The addition of Bovine Dectin-1 CRD-Fc significantly improved the phagocytosis of Alexflour® labeled Zymosan BioParticles® over Fc fragment alone in a concentration dependant manner. See FIG. 6.

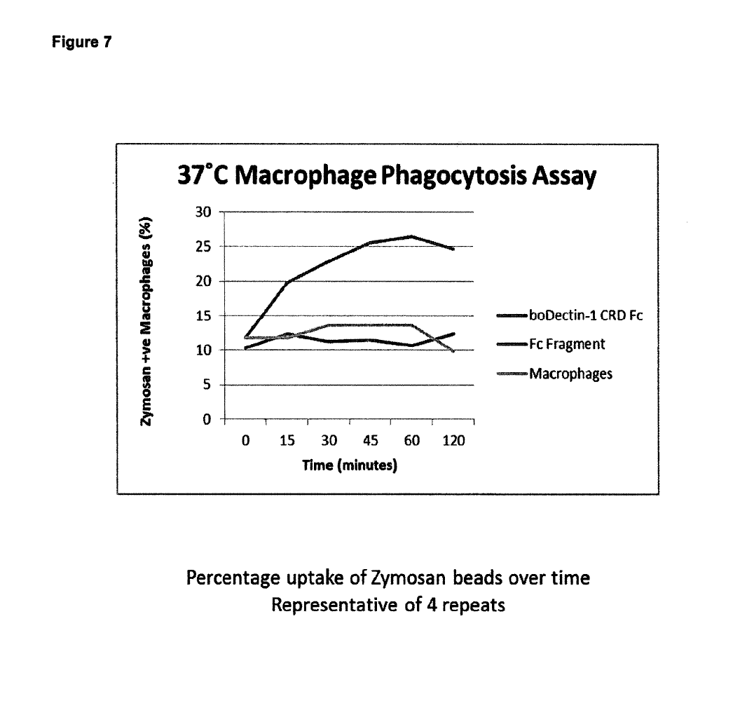

Bovine Dectin-1 CRD-Fc enhances phagocytosis in a time dependant manner when compared to both Fc fragment and macrophages alone. See FIG. 7.

### Summary

Bovine Dectin-1 CRD-Fc fusion vector has been successfully created and expressed.

Bovine Dectin-1 CRD-Fc opsonises Zymosan BioParticles® increasing their phagocytosis in a concentration and time dependant manner.

Further work can include looking at the processing of BioParticles® via confocal microscopy, checking for co-localisation with early and late endosomes

### Example 2

### Assessing the Ability of the Fusion Protein to Activate the Classical Complement Pathway

Bovine Dectin-1 CRD-Fc . Does it activate complement?

### Background

Bovine Dectin-1 is a pattern recognition receptor (PRR) of the innate immune system. It is known to recognise pathogen associated molecular patterns (PAMPs) specifically s glucans found on yeasts and some streptococci and staphylococci. By combining the carbohydrate recognition domain (CRD) of bovine Dectin-1 with the Fc fragment of a human IgG1 antibody we are able to exploit the broad pathogen binding ability of Dectin-1, while targeting the pathogen for phagocytosis via Fc. receptors.

Another important method of pathogen opsonisation for phagocytosis is complement activation. The classical pathway is initiated by binding of complement component C1q to the CH2 domain of an antibody. Bovine Dectin-1 CRD-Fc retains its CH2 binding site so it is hypothesised that complement activation can occur via this construct.

The potential of the bovine Dectin-1 CRD-Fc construct to activate the complement cascade was tested in both a non-physiological and physiological manner. The Dectin-1 construct was compared to Fc fragment alone and controls to give an overall picture of activation.

### Solid Phase ELISA

Complement activation of various test proteins was compared to activation by the protein, human serum albumin (HSA) and an IgG complex. HSA is a normal protein present in human blood and therefore should not activate complement itself, thereby providing us with a background value for complement activation. IgG complex acts as a positive control as a known activator of the classical complement cascade. By comparing the amount of complement components bound to the test proteins, the effect on complement activation can be determined.

### Method

Dilute the test proteins in gelatine veronal buffer (GVBS) and 100 μl placed into duplicate wells for each complement component to be tested on Greiner medium binding ELISA plates (medium binding plates are essential to avoid background complement activation)
Incubate the plate at 4.0 overnight to allow proteins to bind
Wash the plate 3 times with 250 μl PBS/Tween 0.1% per well
Block the plate with 300 μl PBS/Gelatine 0.1% for 1 hr at room temperature (RT)
Wash the plate 3 times with 250 μl PBS/Tween 0.1% per well
Incubate the wells with 50 μl 1/10 normal human serum diluted in GVBS+Ca++/Mg++ or 50 μl 1/10 normal human serum diluted in GVBS +20mM EDTA for 30 minutes at 37.0
Wash the plate 3 times with 250 μl PBS/Tween 0.1% per well
Dilute the primary antibodies in GVBS and place 50 μl in the relevant wells, incubate for 1 hr at RT
Goat anti-sera to human C3 (Quidel) 1:1000
Biotinylated monoclonal anti human iC3b (Quidel) 1:200
Biotinylated chicken anti human C4 (Cedarlane) 1:2000
Mouse monoclonal anti human C5b-9 (Antibody Workshop) 1:200
Wash the plate 3 times with 250 μl PBS/Tween 0.1% per well
Dilute the secondary antibodies in GVBS and place 50 μl in the relevant wells, incubate for 1 hr at RT
Goat anti mouse peroxidase labeled (50:50 in glycerol) 1:1000
Rabbit anti goat peroxidase labeled (50:50 in glycerol) 1:1000
Streptavidin peroxidase labeled 1:500
Wash the plate 3 times with 250 μl PBS/Tween 0.1% per well
Add 50 μl prepared OPD (Dako) (2×2 mg tablets in 6 ml H₂O plus 10 μl 30% H2O2) incubate for up to 15 minutes until developed
Stop the reaction with 50 μl 0.5M H2SO4 (in the same order as OPD)
Read the plate at 492 nm

### Results

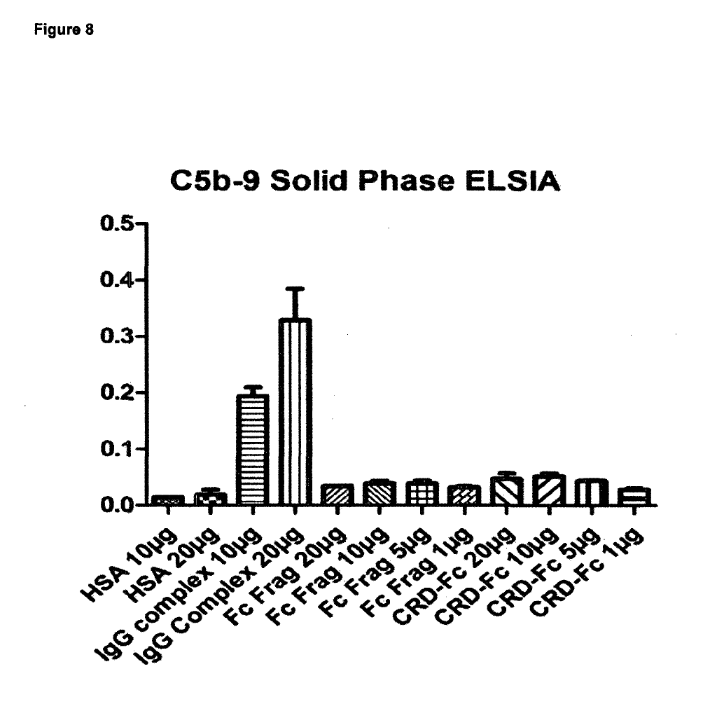

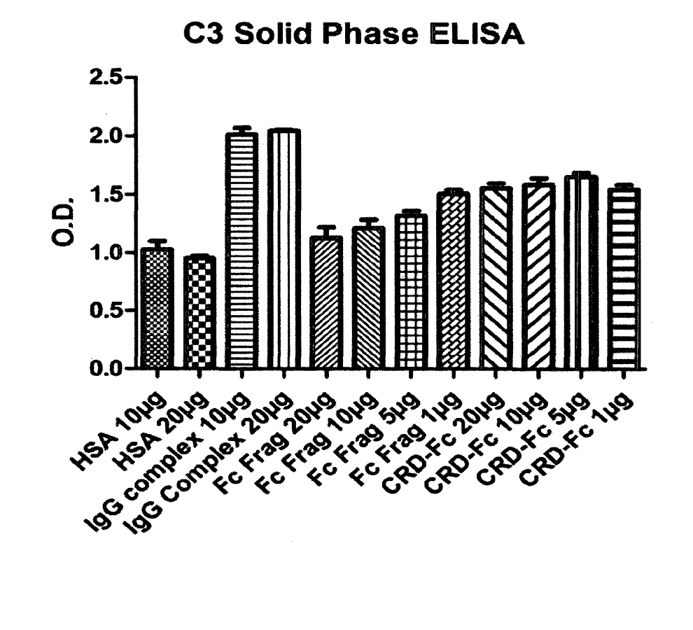

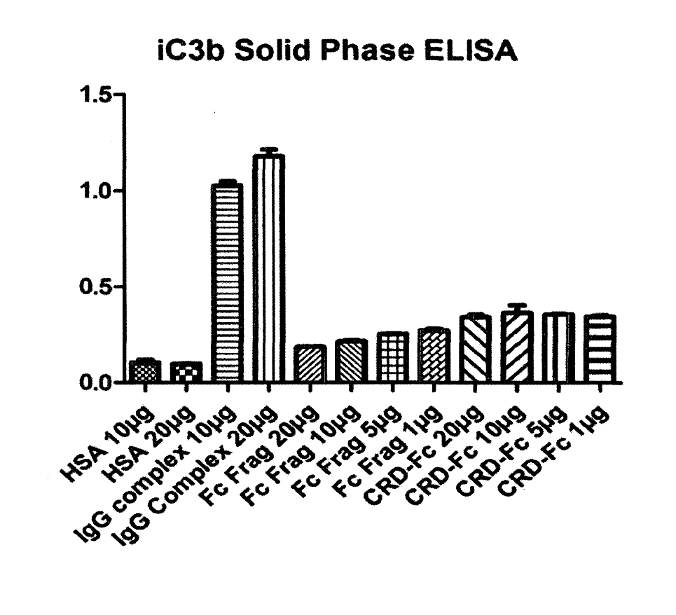

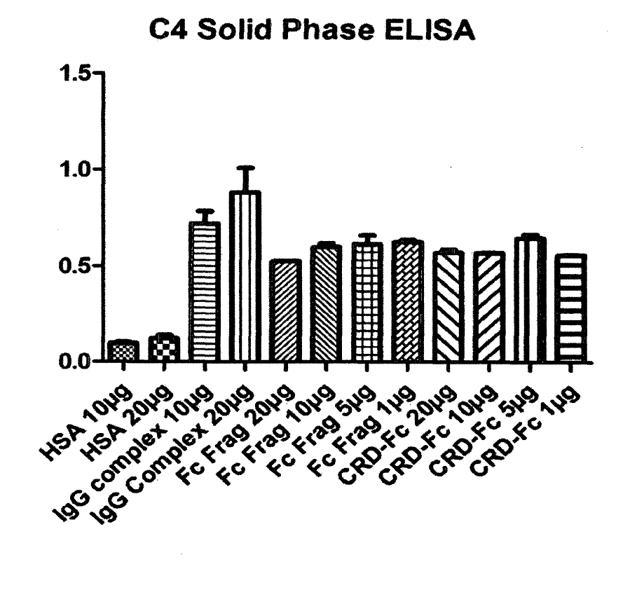

Both positive control, IgG and negative control, HSA worked as expected. O.D. values were compared relative to HSA and analysed using a one way ANOVA (FIG. 1). A concentration dependant effect was not observed with bovine Dectin-1 CRD-Fc for any complement component tested. This could be due to the binding capacity already being exceeded at 1 μg. Future experiments using lower concentrations could elucidate this anomaly. In contrast, Fc fragment alone did show a sight concentration dependent effect. FIG. 8. Complement activation of C3, iC3b, C4 and C5b-9 by solid phase ELISA. HSA negative control and IgG complex positive control. Significance values given are compared to HSA 20 μg. P values *=<0.05**=<0.01***=<0.001

### Fluid Phase Assays

Fluid phase assays were performed with the objective of corroborating and expanding on data gathered from solid phase assays. Human serum albumin was used as a negative control and Zymosan BioParticlesR as a positive control.

### Common Method

Dilute the proteins in GVBS+Ca++/Mg++ or GVBS 20 mM EDTA at double final concentration to a total volume of 100 μl
Dilute normal human serum ⅕ in GVBS+Ca++/Mg++ or GVBS 20 mM EDTA
Add 100 μl of diluted serum to each pre-diluted protein (with and without EDTA)
Incubate the samples for 1 hr at 37.0
Stop the sample reactions with 20 μl 100 mM EDTA stop solution
Dilute the samples to a final volume of 1000 μl with GVBS 20 mM EDTA (final dilution of normal human serum 1:25)
Dispense the samples into five 200 μl aliquots and store at −80.0 for future use CHSO Assay Method
For CH50 assays only the first 3 steps were the same as the common method
Incubate the samples for 2 hr at 37. C
Place the samples on ice
Dilutions of 1:20, 1:40, 1:80, 1:160 normal human serum were made for each sample in 400 μl GVBS +Ca++/Mg++
Add 200 μl antibody coated sheep erythrocytes
Controls 400 μl GVBS +Ca++/Mg++ and 200 μl erythrocytes, 400 μl H₂O and 200 μl erythrocytes, 400 μl H₂O and 100 μl erythrocytes were used
Incubate the samples for 45 min at 37. C in a shaking water bath
Spin the samples at 3000 rpm, 5 min, 4. C
Transfer 200 μl of each supernatant to a flat bottom ELISA plate and measure the O.D. at 540 nm

### Terminal Complement Complex Assay Method

Frozen aliquots from the common method were used for analysis
Terminal complement complex (TCC) assay was performed as per standard diagnostic assay (see appendix for protocol) at 1:25 and 1:50 dilutions
O.D. from results were correlated with the standard curve to provide figures in ng/ml C3a-desArg ELISA
Frozen aliquots from the common method were used for analysis
C3a-desArg ELISA (Progen, Germany) was performed as per the manufactures instructions (see www.progen.de)
Frozen samples were diluted to 1:7500 normal human serum to provide results in the reference range of the standard curve

### Results

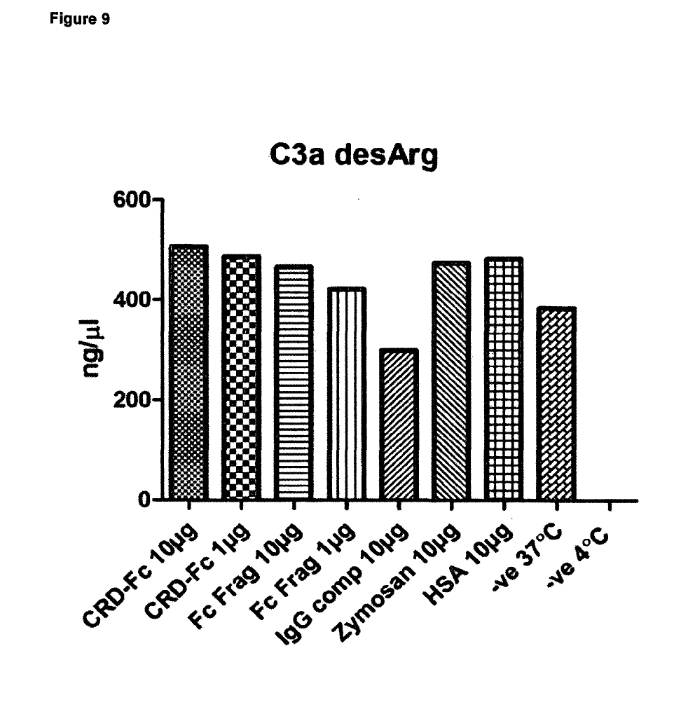

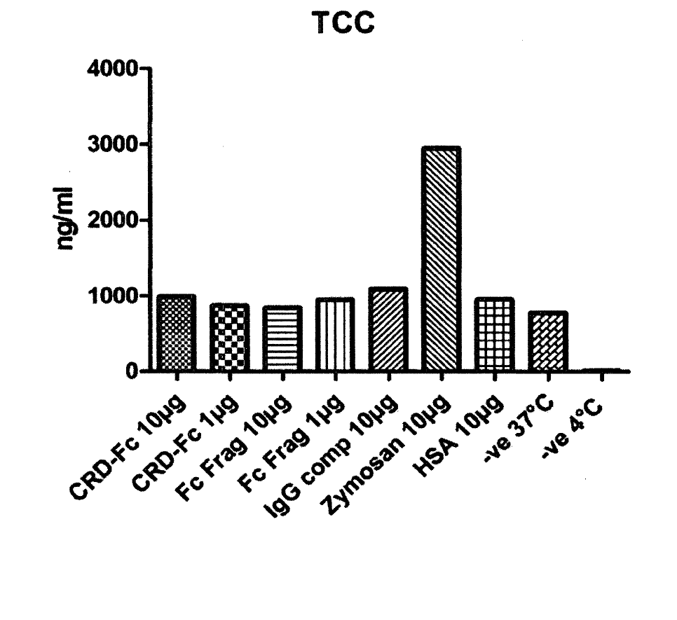

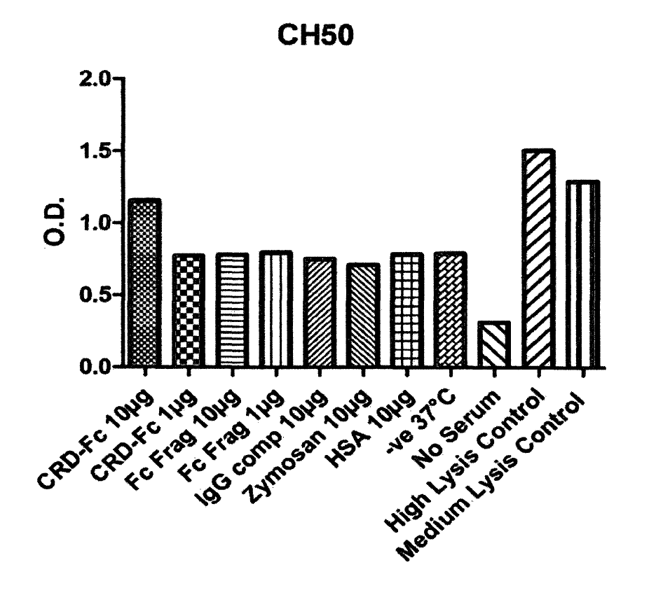

The CHSO assay showed no consumption of complement components, this would have resulted in a reduction of the O.D. value compared to negative control HSA. The C3a-desArg ELISA also showed no difference between positive and negative control, this is likely due to the dilution factor required for this assay. The TCC assay showed a marked response for the positive control. All other samples showed no response over the negative control HSA. This is consistent with the data from the solid phase ELISA. FIG. 9. Complement activation by various proteins tested using fluid phase assays. Zymosan and IgG complex act as positive controls for complement activation and HSA acts as the negative control for background complement activation.

### Physiological Assay

While experimental assays in solid and fluid phase can help determine complement activation, these systems are artificial and do not always correlate to activation in a physiological environment. To confirm the results of previous experiments, Zymosan BioParticlesR were used as artificial pathogens for bovine Dectin-1 CRD-Fc binding. The binding of complement activation products on Zymosan BioParticlesR was measured using flow cytometry.

### Method

For each complement component to be tested make 6 wells as follows in a 96 well “V” bottomed plate;
4 μl 20 mg/ml Zymosan BioParticlesR, up to 50 μl with RPMI
4 μl 20 mg/ml Zymosan BioParticlesR, 10 μg/ml Fc Fragment, up to 50 μl with RPMI 20 mM EDTA
4 μl 20 mg/ml Zymosan BioParticlesR, 10 μg/ml bovine Dectin-1 CRD-Fc, up to 50 μl with RPMI 20 mM EDTA
4 μl 20 mg/ml Zymosan BioParticlesR, bovine Dectin-1 CRD-Fc
4 μl 20 mg/ml Zymosan BioParticlesR, 10 μg/ml Fc Fragment, up to 50 μl with RPMI
4 μl 20 mg/ml Zymosan BioParticlesR, 10 μg/ml bovine Dectin-1 CRD-Fc, up to 50 μl with RPMI
Incubate the plate for 1 hr at 37. C
Add 50 μl normal human serum ⅕ in GVBS+Ca++/Mg++ or GVBS 20 mM EDTA
Incubate the plate for 1hr at 37. C
Add 10 μl 100 mM EDTA stop solution and incubate for 5 minutes
Spin the plate at 1000 rpm, 3 minutes, then remove the supernatant
Re-suspend the beads with 200 μFACS buffer (PBS/1% BSA/0.05% Azide)
Spin again a further 2 times
Resuspend the Zymosan BioParticlesR in 25 μl relevant primary antibody in FACS buffer;
Biotinylated monoclonal anti human C1q (Quidel) 1:50
Goat anti-sera to human C3 (Quidel) 1:100
Biotinylated monoclonal anti human iC3b (Quidel) 1:50
Biotinylated chicken anti human C4 (Cedarlane) 1:200
Mouse monoclonal anti human C5b-9 (Antibody Workshop) 1:50
Incubate the plate for 15 minutes at RT
Wash the plate 3 times as before
Resuspend the Zymosan BioParticlesR in 25 μl relevant secondary antibody in FACS buffer;
R-PE anti Biotin Fab Fragment 1:100
FITC anti mouse Fab fragment (1:1 Glycerol:Antibody) 1:50
FITC anti goat Fab fragment (1:1 Glycerol:Antibody) 1:50
Incubate the plate for 15 minutes at RT
Wash the plate 3 times as before
Resuspend each sample in 100 μl FACS buffer and transfer to a labeled FACS tube containing 300 μl FACS buffer
Samples were analysed on the FACS Calibur (Becton Dickinson) counting 20,000 events for each sample.

### Results

Complement components C1q, C3, iC3b, C4 and C5b-9 were tested for complement binding and activation. Activation of complement was compared between Zymosan BioParticlesR alone or with the addition of either Fc Fragment or bovine Dectin-1 CRD-Fc. Components C1q, C3 and C4 all show a trend to increased activation, although there is no overall significance compared to Zymosan BioParticlesR alone when analysed by one way ANOVA. C5b-9 correlates with both solid and fluid phase assays confirming that bovine Dectin-1 CRD-Fc does not result in the formation of the membrane attack complex.

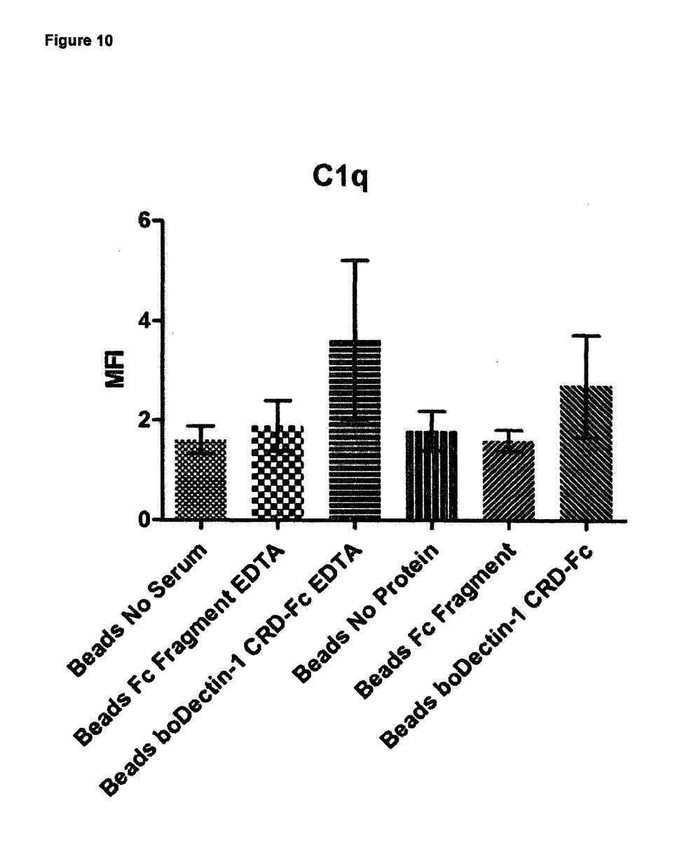

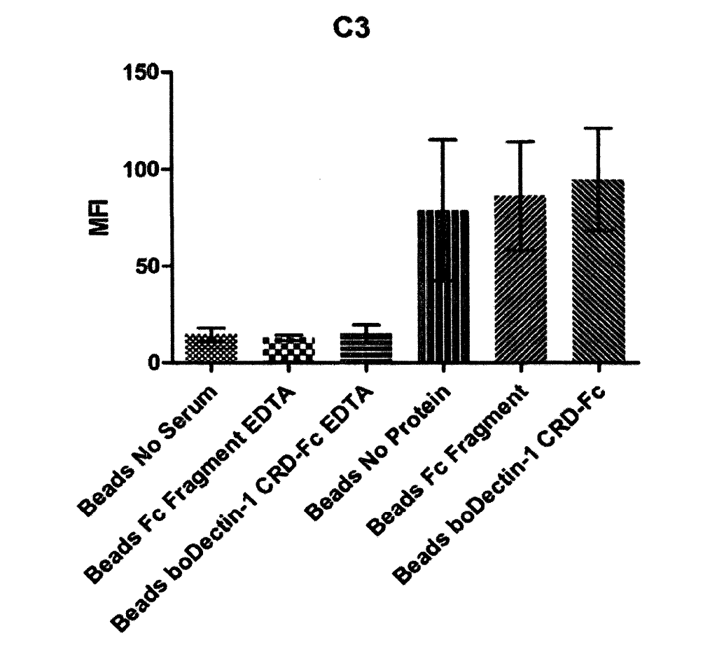

FIG. 10. Physiological complement activation by Zymosan BioParticlesR analysed by flow cytometry. Complement components bound to Zymosan BioParticlesR were compared with and without treatment with Fc Fragment and bovine Dectin-1CRD-Fc. No serum and EDTA repeat act as controls.

### Discussion

Each of the assays performed looked at the activation of the complement cascade and subsequent deposition and formation of complement components. The individual assays have both positives and negatives, so only by comparing complement activation over all the assay systems can an accurate picture be derived.

The only assay system to provide results which gave significant results compared to the negative control was the solid phase ELISA. Initially, problems with this system came from high background. However, this was resolved with the use of medium binding ELISA plates. The binding of protein to the plate in this system artificially simulates the effect of bovine Dectin-1 CRD-Fc binding to a pathogen. This, therefore, poses the question as to how applicable this assay is to complement activation in a physiological environment.

Fluid phase assays went some way to try and close the gap between physiological and artificial activation. Looking at the CHSO assay which grossly looks at complement deficiency, the proteins were unable to consume enough complement to cause any change in the CHSO value. This could be due to the fact that in this system, bovine Dectin-1 CRD-Fc isn't bound to a pathogen so, therefore, does not activate the classical cascade. Another factor could be related to the quantities that were used. Complement proteins are very abundant in serum and far higher concentrations of test proteins may be required to cause a change in this value. While this result does not help elucidate complement activation, at concentrations likely to be used in vivo, this result shows bovine Dectin-1 CRD-Fc is less likely to have adverse effects such as complement activation and depletion. The C3a-desArg assay proved to be inadequate for the experimental design. Even at background levels the formation of C3a-desArg was at far higher levels than the ELISA was designed to cope with. Subsequently the high dilution necessary to bring the O.D. into the middle of the standard curve has eliminated any possible differences. This ELISA would need to be re-designed to allow accurate measurement of this component. The TCC assay is a standard ELISA used for human diagnostics. This assay performed well in conjunction with the fluid phase samples giving meaningful results, which correlated with results from the solid phase ELISA.

The physiological assay attempted to replicate as close as possible the natural pathway of complement activation. Bovine Dectin-1 CRD-Fc binds Zymosan BioParticlesR, therefore providing a more realistic model for complement activation. A drawback of Zymosan BioParticlesR is they are known activators of the alternative pathway, meaning any differences observed are likely to be small. In the experiments described here, Zymosan BioParticlesR activated complement without opsonisation. Although there was no significance in any of the results there is a clear trend to weak activation of the classical complement cascade. These experiments could be repeated with Factor B deficient serum, which would help give a clearer picture by knocking out the alternative pathway.

Taking the results together from all three assays, bovine Dectin-1 CRD-Fc promotes weak activation of the classical complement pathway upon binding to a pathogen. In all experimental models shown here this does not result in the formation of the terminal complement complex C5b-9. Therefore, any positive effect on pathogen clearance would rely on the opsonising effect of bound complement components and the production of anaphylatoxin to cause phagocytosis via receptors such as CD11b.

### Example 3

### Mannose/Mannan Binding Lectin (MBL) Constructs

Human mannose/mannan binding lectin (MBL; also MBPC) is a 25 kDa member of the collectin family of pattern recognition molecules. Human MBL is 63%, 61% and 65% aa identical to mouse, porcine and bovine MBL, respectively [1, 2]. MBL has been show to bind to yeasts such as Candida albicans, viruses such as HIV and influenza A, many bacteria including Salmonella, Staphylococci, Streptococci, Actinobacilli and Haemophilus parasuis, as well as parasites like Leishmania [1, 3-5]. It is a secreted glycoprotein that is synthesized as a 248 amino acid (aa) precursor that contains a 20 aa signal sequence, a 21 aa cysteine-rich region (with three cysteines) a 58 aa collagen like segment and a 111 aa C-type lectin domain that binds to neutral bacterial carbohydrates [6]. The molecule is O glycosylated and contains multiple hydroxylated prolines and lysines. Functionally, the molecule operates as a multimer/oligomer. The basic structural unit is a homotrimer [7]. The homotrimer is created by the formation of interchain-disulfide bonds among the cysteine rich regions, plus a helical interaction of the collagen like domains of each participating polypeptide. Mutations in the collagen region are known to interfere with proper trimer and subsequent oligomer formation. Once formed, the trimer, as a unit, oligomerizes with other trimers to form high molecular weight complexes. Although the exact nature of these complexes is unclear, it would appear that a three trimer complex (230 kDa) and a four trimer complex (305 kDa) constitute much of the circulating MBL [7]. It is within the context of these oligomers that MBL performs its functions. After secretion by hepatocytes, oligomerized MBL will both associate with serine proteases (MASP1, 2 & 3) and bind to bacterial carbohydrates [8]. Binding of MBL to a microorganism results in activation of the lectin pathway of the complement system. If the MBL complex is small, opsonisation of bacteria occurs. If the complex is large, the MASPs are engaged and a complement attack complex is generated, destroying bound bacteria. Here, in order to activate the complement system when MBL binds to its target (for example, mannose on the surface of a bacterium), the MASP protein functions to cleave the blood protein C4 into C4a and C4b. The C4b fragments can then bind to the surface of the bacterium, and initiate the formation of a C3 convertase. The subsequent complement cascade catalysed by C3 convertase results in creating a membrane attack complex (MAC), which causes lysis of the pathogen that MBL bound to [8-11]. Another important function of MBL is that this molecule binds senescent and apoptotic cells and enhances engulfment of whole, intact apoptotic cells, as well as cell debris by phagocytes [12].

We have recently cloned the MBL in the bovine system. See Example 5 below. We consider that soluble chimeric proteins consisting of the carbohydrate recognition domain of a MBL and the Fc-part of an IgG molecule enhance phagocytosis of bacteria by macrophages, neutrophils and dendritic cells, as well as stimulating all arms of the complement cascade. The potential of this artificial opsonin to enhance bacterial killing can be assessed by:

1) Cloning, sequencing and expressing CRD domains of human/bovine mannose-binding lectin as Fc-tagged proteins using Fc fragments from different species and different isotypes
2) Assessing the functionality of CRD-Fc proteins by assessing their ability to bind Gram-positive bacteria in an competitive ELISA-like assay
3) Assessing the functionality of CRD-Fc proteins by assessing their ability to opsonise bacteria and to induce increased phagocytosis in different cell-types in vitro
4) Assessing the ability of the created artificial opsonins to enhance clearance of S. aureus induced systemic and local infection in mice
1. Cloning, sequencing and expressing CRD domains of human/bovine/ovine mannan-binding lectin (MBL) as Fc-tagged proteins using Fc fragments from different species and different isotypes.

The sequence of the bovine mannose-binding lectin has been identified (see Example 5), while sequences of the ovine and human orthologues are already known [2, 13]. The CRD of MBL can be cloned into the pSecTag mammalian expression vector (Invitrogen Life Technologies), containing the IgK leader sequence facilitating protein secretion. Once cloned, the MBL-CRD can be cloned with different Fc parts of IgG subclasses. For MBL-Fc protein generation, HEK293 cells can be transiently transfected with the constructs pSecTag 2C-CRD-Fc vector, and supernatants can be harvested. The secreted protein can be affinity purified over a column consisting of Sepharose beads conjugated to goat-anti-mouse H chain IgG (Sigma-Aldrich), and purity can be checked by SDS-PAGE followed by Coomassie Blue staining.

2. Assessing the functionality and specificity of CRD-Fc proteins in an competitive ELISA-like assay

To assess the binding capacity of the MBL-Fc fusion proteins to different bacteria, as well as to assess binding-specificity to bacteria via the MBL-CRD part, an ELISA-type system can be used. To do so, the MBL-CRD-Fc protein can, for example, be absorbed onto 96-well microtiter plates. After incubation and washing, life Gram-positive bacteria can be incubated in various concentrations, before counterstained using a second, bacteria-specific antibody. The bound complex can subsequently be visualised using a HRP-coupled third antibody, following enzymatic reaction and measurement of absorbance using an ELISA-plate reader. To assess binding specificity, each reaction can be incubated with increasing concentrations of recombinant mannan (R&DSystems) to compete for bacterial binding. In addition, where possible, bacterial strains can be assessed that either lack protein A expression (to assess further specificity of binding through MBL, and not through the Fc part of the MBL-CRD-Fc fusion protein) and the results compared to wild-type strains.

3. Assessing the functionality of MBL-CRD-Fc proteins by assessing their ability to increase phagocytosis in different cell-types as well as to activate the complement system

Bacterial organisms are identified to bind to the ELISA-system described before. These bacteria can be incubated with the MBL-CRD-Fc protein before being added to phagocytes. Where possible, uptake of bacteria can be assessed by flow cytometry using bacterial strains expressing fluorescent dyes, such as FITC-labeled S. aureus CP5. Uptake can be monitored over time, using different concentrations of the MBL-CRD-Fc fusion protein, fusion proteins expressing different Fc part, and at different temperatures (4° C. and 37° C.). To assess specificity of uptake, some groups can be pre-treated with either (bovine) serum (40% vol/vol in HBSS) to assess Fc receptor mediated phagocytosis, recombinant mannan (to block the MBL-CRD)) and/or bovine specific antibodies to FcγRII/III. As a control, FITC-zymosan (Molecular Probes) can be used. This can be enumerated and preopsonised with conditioned supernatant containing CRD-Fc. Cells can be distinguished from free bacteria/zymosan by forward and side scatter profiles, as well as by quenching using 0.04% Trypan Blue. Mean fluorescence intensity (MFI) can be calculated by averaging all events across the live cell gate; fold changes are calculated by normalizing the observed MFI to the baseline MFI obtained from cells incubated with zymosan preopsonised with control medium. Flow cytometric assays can be performed using a FACSCalibur™ (BD Biosciences), and results can be analysed using FlowJo™ software.

In addition to the phagocytosis-assay, it can also be investigated whether the complement-activation centre within the Fc part, as well as the lectin-dependent pathway of complement activation are still functional in the constructs, for example using techniques as described in Example 2 above.

4. Assessing the ability of the created artificial opsonins to enhance clearance of S. aureus induced systemic and local infection in mice.

To assess the potential of the MBL-CRD-Fc protein to help clearance of a bacterial infection, two different infectious S. aureus models can be used.

1) Assessment of the effects of the MBL-CRD-FC protein to a systemic S. aureus infection

To do so, MBL -/- mice (commercially available) can be infected with the bioluminescent S. aureus Xen 8.1 (biolumi-S. aureus; Caliper Life Sciences, USA), which is a modification of S. aureus 8325-4. This biolumi-S. aureus can be used for studies of in vivo imaging. Mice can be inoculated i.v. in the tail vein with different concentrations of the strain. After different time points, mice can be reconstituted, with different dosages of the MBL-CRD-FC fusion protein to achieve a range of 5 to 11 μg/ml MBL, which is in the physiological range in mouse. In Vivo Bioluminescence Imaging can be performed using a low light imaging system (Hamamatsu Photonics KK, present at the London

School of Hygiene and Tropical Medicine). In addition, bacterial load in blood and organs can be assessed by blood sampling from the tail-vein as well as harvesting organs from killed mice. Organs can be weighed, homogenised, and serial dilutions of the blood and the organ homogenates can be cultured on tryptic soy agar plates supplemented with 5% sheep blood plates (TSA-II) overnight at 37° C. CFUs can be calculated as CFU/ml for blood and CFU/g of wet weight for organs.

2) Assessment of effects of the MBL-CRD-FC protein in a local mammary gland S. aureus infection. See, for example, Example 4.

### Example 4

### In Vivo Assessment

The inventors have cloned several CRDs from bovine receptors, and have linked these to the constant region (Fc) of an antibody. By doing so, the inventors consider that pathogens are bound in a specific manner through the CRD, whereas the Fc part targets the bound (“opsonised”) pathogens to phagocytosis-active cells, such as macrophages/granulocytes, as well as activating the complement system through sites present in the Fc part. Both of these effects have been tested already in vitro, and we now extend the studies into in vivo analysis, infusing the artificial opsonin into affected udder quarters, alongside the normal antibiotic therapy.

We provide a new treatment strategy, using a combination therapy including the artificial opsonin with an antibiotic treatment. Having already established that one construct increases phagocytosis of pathogens in vitro, further work and the in vivo trial aim to confirm that the same occurs for a construct directed against St. aureus sugar moieties, and that the construct reduces significantly the bacterial load in St. aureus infected udders without inducing unwanted side-effects, such as an overshooting immune response. The construct effect against other Gram-positive Mastitis-causing bacteria, such as Streptococcus uberis/agalactiae/dysgalacectiae can also be assessed. Given the fact that the sugar moieties expressed by all of these bacteria are similar, the construct is considered very likely to be effective against such bacteria, confirming wide applicability.

We have already produced two fusion proteins containing the Fc region of IgG1 linked with the CRD of dectin-1 and Mannose-binding protein. The Dectin-1 CRD-Fc fusion protein has shown an increased phagocytosis of yeast. The MBL CRD-Fc fusion protein is considered to be more suitable for use with Gram-positive bacteria.

Assessment of this construct is described in, for example, Example 3 above. In summary, the mannose binding lectin construct is considered to bind specifically to St. aureus and other Gram-positive bacteria. The dose-response, time-response, temperature-response of this construct in enhancing the phaogcytosis of a dye-labeled St. aureus by milk- and blood-derived macrophages can be analysed. Once confirmed as effective, different Gram-positive bacteria strains isolated from clinical mastitis cases can be tested, to confirm the general application of the product.

A small-scale in vivo trial is planned to assess the potential safety risks of infusing the protein into the udder. Within the bovine system, the udder comprises 4 independent quarters, allowing for a whole set of exposures run within one udder. Udders of 6 cows will be treated with the fusion protein or the Fc part of the fusion protein alone before subsequent exposure to St. aureus, only infected or left uninfected, untreated. Clearance of bacteria will be assessed over a 48 hr period, and immune parameters, such as cytokines will be analysed in the milk. Other Gram-positive, Mastitis causing bacteria, such as Strep. Uberis/Strep. Agalactiae/dysgalactiae can also be tested in the in-vitro system specified.

### Example 5

### Bovine MBL Sequences and Exemplary Sequence of a Compound Comprising a C-type Lectin Carbohydrate Recognition Domain (CRD) and an Immunoglobulin Fc Domain

Bovine MBL was sequenced and the CRD sequence used in forming constructs.

Bovine MBL sequences are also published: locus NM_174107. See, for example http://www.ncbi.nlm.nih.gov/nuccore/NM 174107.2:

gccctggtga ggatcatgtc gctgtttaca tcacttcctt
ttcttctcct gactgcggtg acagcatctt gtgcagacac
agaaacagag aactgtgaga acatccggaa gacctgcccc
gtgattgcct gtggtcctcc gggcatcaat ggcatcccag
gcaaagatgg gcgtgatggt gccaagggag aaaagggaga
accaggtcaa ggactcagag gctcgcaggg cccccctgga
aagatggggc ctcaaggaac gccagggatc cctgggatac
caggaccaat aggccaaaaa ggagaccctg gagaaaatat
gggtgactat attcgcctgg ctacttcaga aagagcaact
ctacaatctg aattgaacca gatcaaaaac tggctaatct
tctctctggg caaaagagtt gggaagaagg cattttttac
caatggtaaa aagatgcctt ttaatgaagt gaagactctg
tgtgcacagt tccagggccg tgtggccacc cctatgaatg
ctgaagaaaa cagggccctc aaggatttag tcactgaaga
ggccttcctg ggcatcacag atcaggagac tgaaggcaaa
tttgtggatc tgacaggaaa gggggtgacc taccaaaact
ggaatgatgg cgagcctaac aacgcttctc ctggggagca
ctgtgtgaca cttctgtcgg acggcacatg gaatgacatc
gcttgttccg cctccttttt gaccgtctgt gaattctctc
tctgagggag aatgagccta aagtcctcct gttcctttac
tcatctcatg ggcccacaac ctggtttgga ggataaatct
atgtcaattt cacacaccca gtactgagtt gctcttttgt
ggggaaacag agacaataag aatggattga gaatgatgag
atgtggaact gaaaagcgtg aagagactta tagtggtgta
agagtttctg gttcagaccc agtcactaat aataatcatt
tcaggaacaa caaaataatg gtagtaatag tagcaacagc
agtaatagtg ggagtactaa taacatattt taaaatgttt
actatgagtc agacattaca tgtaagatta tacattaagt
atctaattta actaggctgt tcatttttgt ttgagactat
aaagaaagct gagcactgaa gaattgatgg ttttgaactg
tggtgttgga gaagactctt aagagtccct tggactgcaa
ggagatccaa ccagtccatc ttaaaggaga tcagtcctag
gtgttcattg gaaggactga agttgaagct gaaactccaa
tactttggcc acctgatgca aagagctgac tcatttgaaa
agaccctgaa gctgggaaag attgagggca ggagaaaaag
ccggaattc
MSLFTSLPFLLLTAVTASCADTETENCENIRKTCPVIACGPPGI
NGIPGKDGRDGAKGEKGEPGQGLRGSQGPPGKMGPQGTPGIPGI
PGPIGQKGDPGENMGDYIRLATSERATLQSELNQIKNWLIFSLG
KRVGKKAFFTNGKKMPFNEVKTLCAQFQGRVATPMNAEENRALK
DLVTEEAFLGITDQETEGKFVDLTGKGVTYQNWNDGEPNNASPG
EHCVTLLSDGTWNDIACSASFLTVCEFSL

MBL precursor

Below is the sequence of bovine (bo) MBL CRD pFUSE C4 pFUSE F

TTTTTTTCTGTTCTGCGGCGTTACAGATCCAGCTGTGACCGGCGCCTACC
TGAGATCACCGGCGAAGGAGGGCCACCATGTACAGGATGCAACTCCTGTC
TTGCATTGCACTAAGTCTTGCACTTGTCACGAATTCGATATCGGCCATGG
TTAGATCTTGGCTAATCTTCTCTCTGGGCAAAAGAGTTGGGAAGAAGGCA
TTTTTTACCAATGGTAAAAAGATGCCTTTTAATGAAGTGAAGACTCTGTG
TGCACAGTTCCAGGGCCGTGTGGCCACCCCTATGAATGCTGAAGAAAACA
GGGCCCTCAAGGATTTAGTCACTGAAGAGGCCTTCCTGGGCATCACAGAT
CAGGAGACTGAAGGCAAATTTGTGGATCTGACAGGAAAGGGGGTGACCTA
CCAAAACTGGAATGATGGCGAGCCTAACAACGCTTCTCCTGGGGAGCACT
GTGTGACACTTCTGTCGGACGGCACATGGAATGACATCGCTTGTTCCGCC
TCCTTTTTGACCGTCTGTGAATTCTCTCTCTTAAGATCTGACAAAACTCA
CACATGCCCACCGTGCCCAGCACCTGAACTCCTGGGGGGACCGTCAGTCT
TCCTCTTCCCCCCAAAACCCAAGGACACCCTCATGATCTCCCGGACCCCT
GAGGTCACATGCGTGGTGGTGGACGTGAGCCACGAAGACCCTGAGGTCAA
GTTCAACTGGTACGTGGACGGCGTGGAGGTGCATAATGCCAAGACAAAGC
CGCGGGAGGAGCAGTACAACAGCACGTACCGTGTGGTCAGCGTCCTCACC
GTCCTGCACCAGGACTGGCTGAATGGCAAGGAGTACAAGTGCAAGGTCTC
CAACAAAGCCCTCCCAGCCCCCATCGAGAAAACCATCTCCAAAGCCAAAG
GGCAGCCCCGAGAACCACAGGTGTACACCCTGCCCCCATCCCGGGGAGGA
GATGACCAAGAACCAGGTCAGCCTGACCTGCCTGGTCAAAGGCTTCTATC
CCAGCGACATCGCCGTGGGAGTGGGGAGGAGCATGGCAGCGGAGACACTA
CAGACCACGCCTCCCGTGCTGGACTCGACGCTCTCTCTCTACAGCAGCTC
ACGTGACAGAGCAGTGCAGCAAGGGACGTCTTCTCATGCTCGTGGATGCA
CGAGTCTGCAACACTTACACCAAGCTACTGTTCTCGGTATGAGTGCTGAC
TGCACGGACTGGGTAGGAAATTACCCCTG

Underlined sequence is the bovine MBL-CRD sequence. The sequence in bold is the human Fc and hinge sequence. The sequence between the underlined (CRD) and bold (Fc/hinge) sequence is a Bg/II restriction enzyme site. The sequence in italics is IL2 secretory leader sequence present in the pFUSE vector. The remaining sequences are also vector-derived.

The same or similar sequences for the CRD and the Fc domain may be used in combination with other CRD or Fc domains, as discussed above.

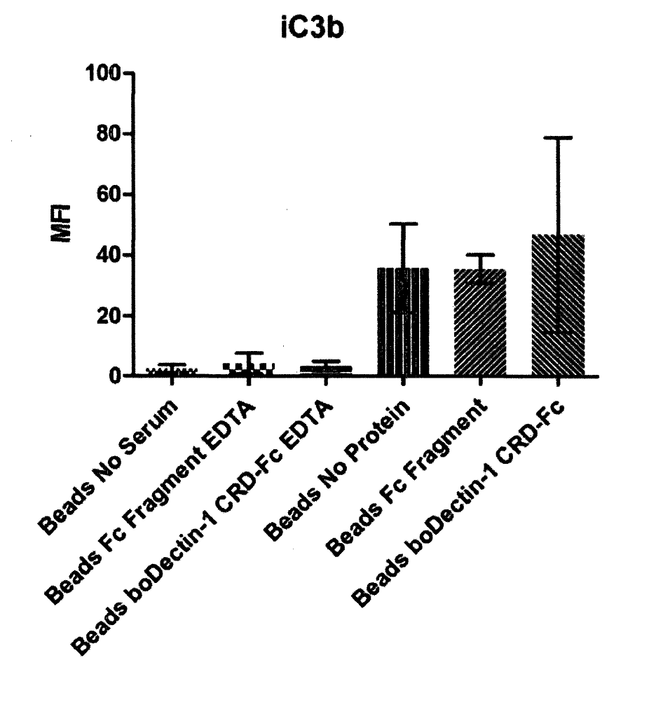

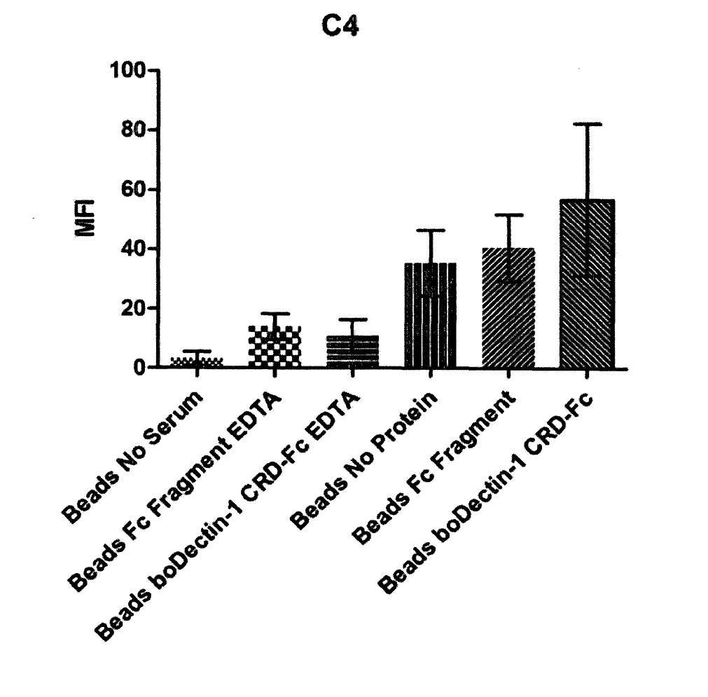

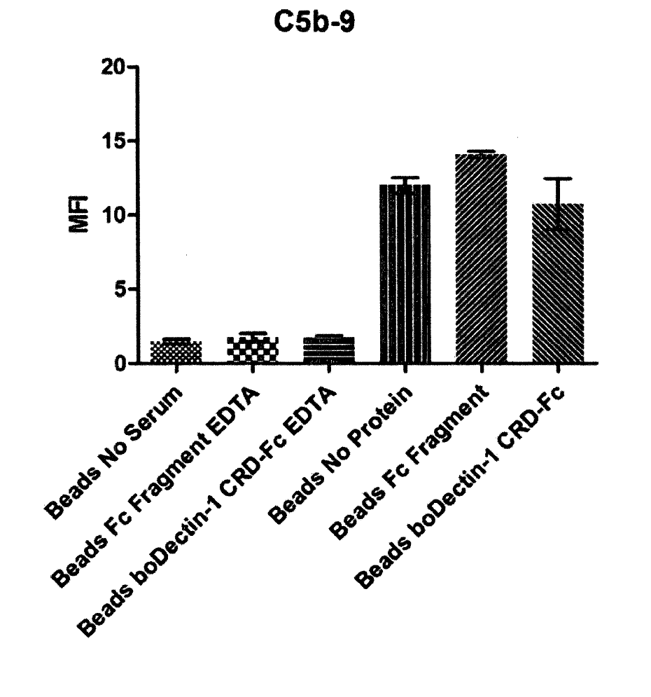

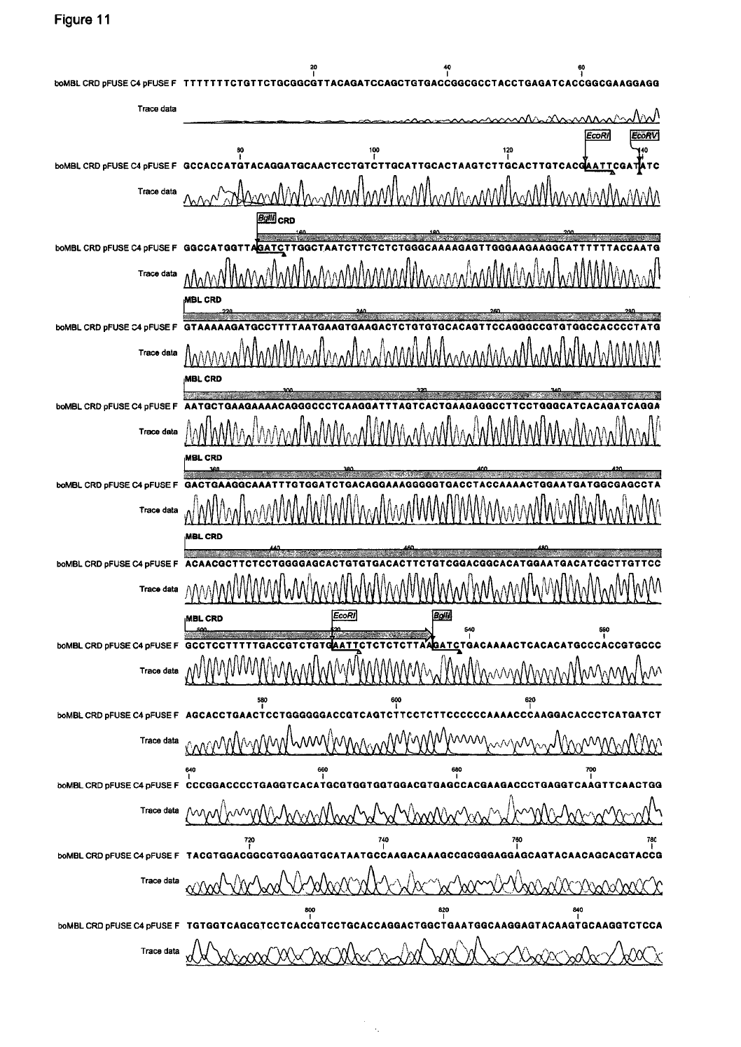

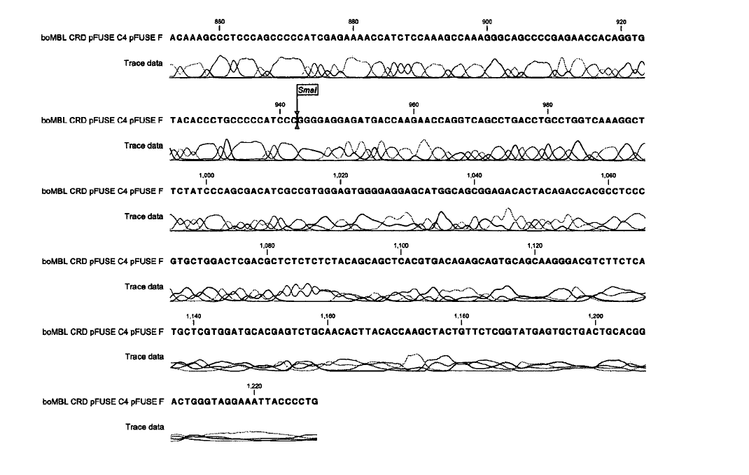

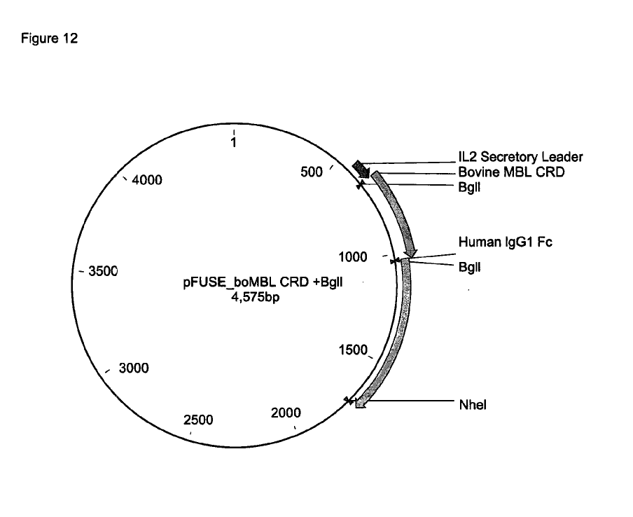

See also FIGS. 11, 12 and 13 which show sequence analysis, a plasmid diagram and sequence confirmation.

### Example 6

### Bacterial Killing Assay

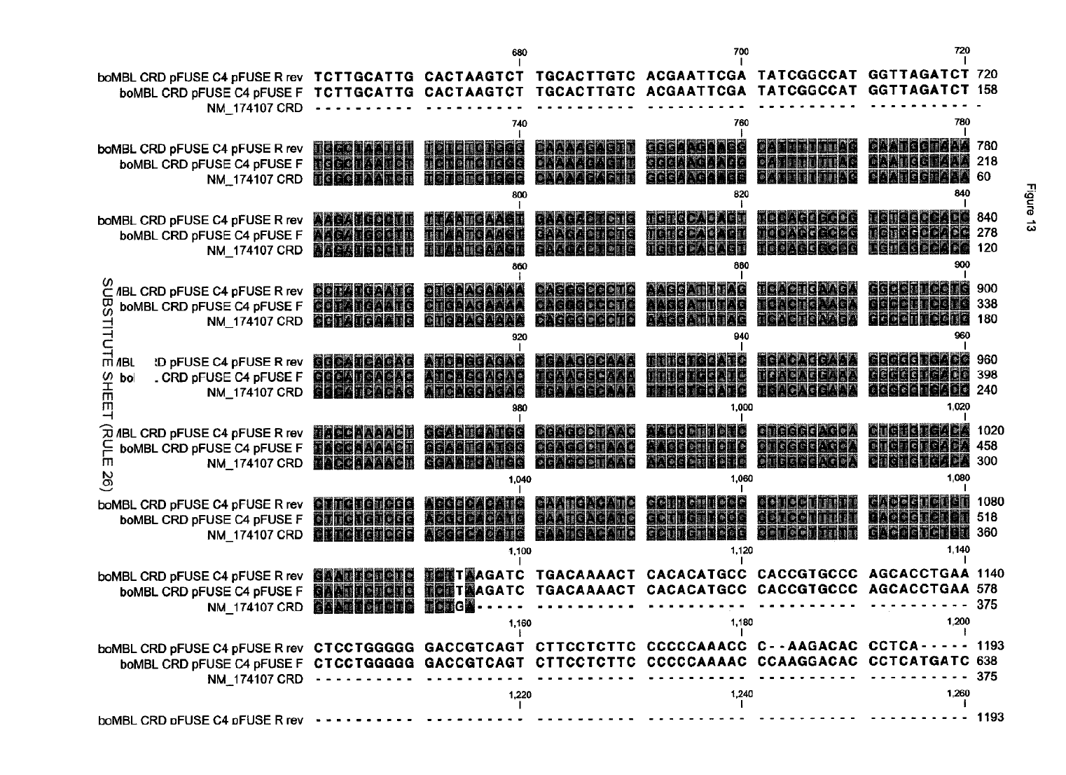

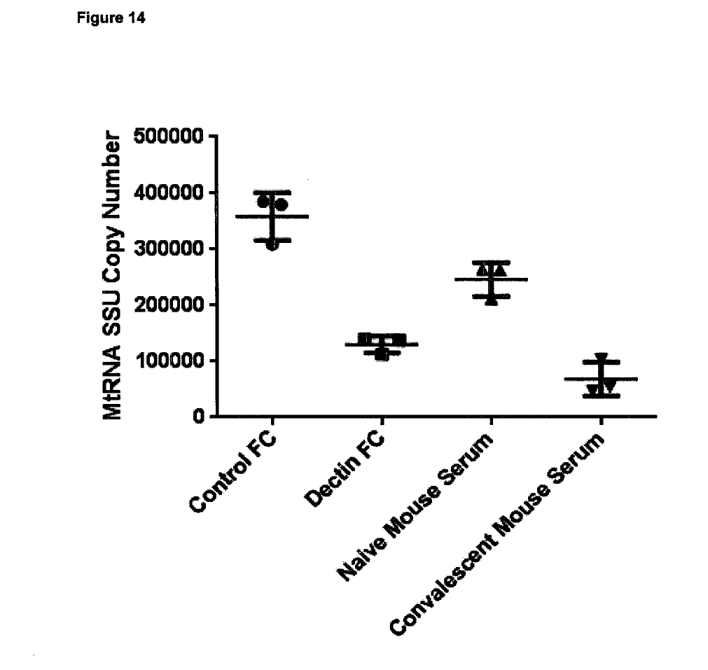

The results shown in FIG. 14 were obtained using with thioglycolate elicited murine macrophages. Fc proteins were used at 0, 5, 10, 50 ug/mL. The protein is a BoDectin-1 CFR Fc construct. Below is the short description of the assay, which is based on:

Steele C, Marrero L, Swain S, Harmsen A G, Zheng M, Brown G D, Gordon S, Shellito J E, Kolls J K. 2003. Alveolar macrophage-mediated killing of Pneumocystis carinii f. sp. muris involves molecular recognition by the Dectin-1 beta-glucan receptor. J. Exp. Med. 198:1677-1688

### Pneumocystis Viability Assay.

P. murina (1×10⁴ asci per well, estimated 1:10 ascus-to-trophic-form ratio) was cultured in 96-well round-bottom plates in DMEM plus 10% fetal bovine serum (FBS). Serum was treated by heat inactivation for 30 min at 56° C. to deplete complement activity (HI FBS) or left untreated (non-HI FBS). P. murina was treated with affinity-purified Dectin-1:Fc at various concentrations and cultured for 24 h. A viability control of P. murina incubated with control medium was included. Following incubation, the contents of the wells were collected and total RNA was isolated using TRIzol-LS reagent (Life Technologies, Carlsbad, Calif.). The viability of P. murina was analyzed with real-time PCR measurement of rRNA copy number as described below.

RNA Isolation and TaqMan Probes and Primers for Pneumocystis rRNA. The assay for determination of P. murina copy number per whole lung has been previously described (Zheng M, Shellito J E, Marrero L, Zhong Q, Julian S, Ye P, Wallace V, Schwarzenberger P, Kolls J K. 2001. CD4+ T cell-independent vaccination against Pneumocystis carinii in mice. J. Clin. Invest. 108:1469-1474). Briefly, cDNA was synthesized with iScript reverse transcription reagents (Bio-Rad, Hercules, Calif.), and real-time PCR was performed using primers for the P. murina large-subunit rRNA gene with SsoFast Probes Supermix (Bio-Rad). The threshold cycle values were converted to rRNA copy number by using a standard curve of known copy number of Pneumocystis rRNA as previously described (Steele et al., 2003, supra).

### Example 7

### Further In Vitro Assays

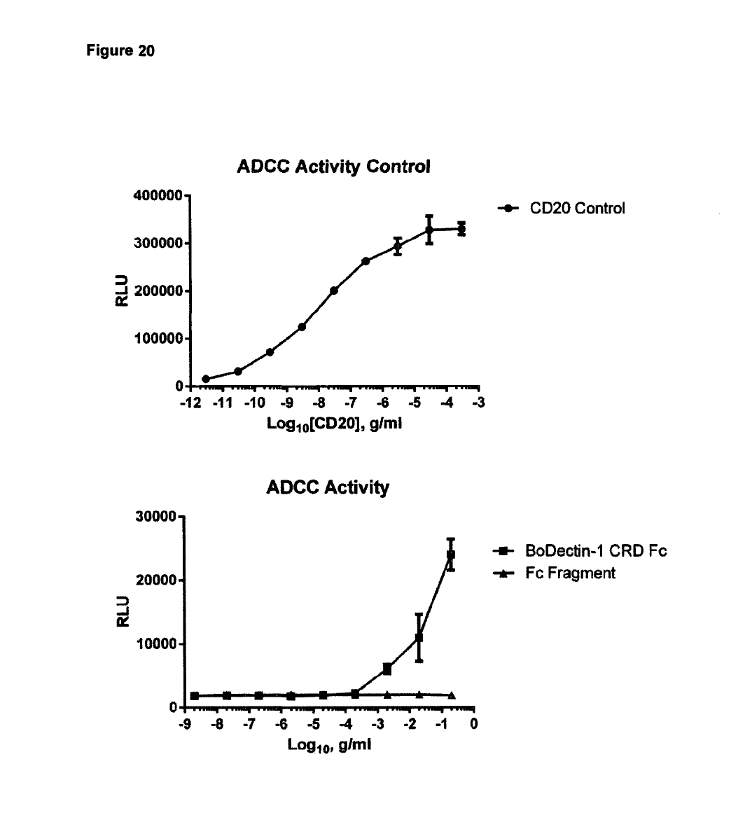

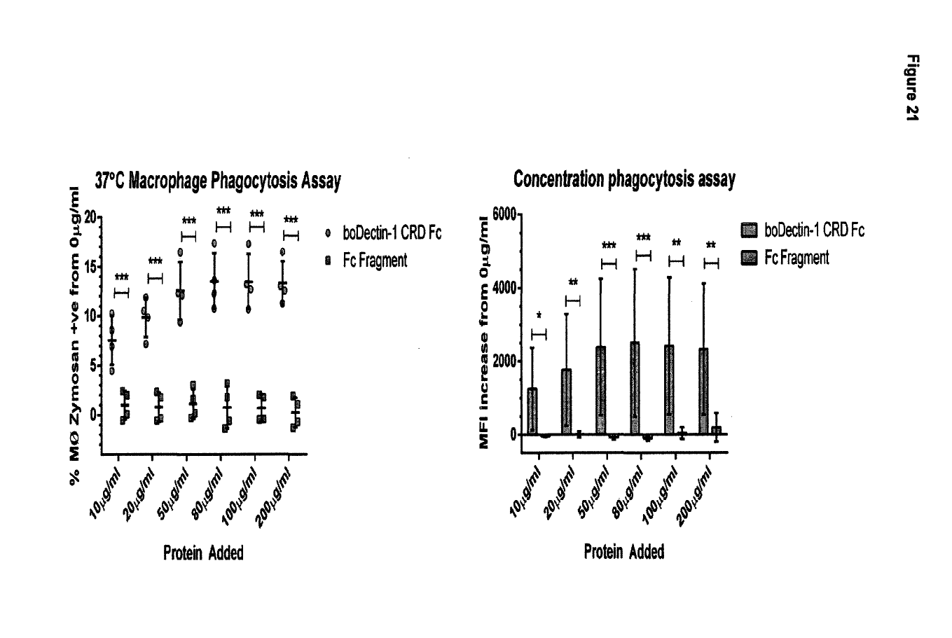

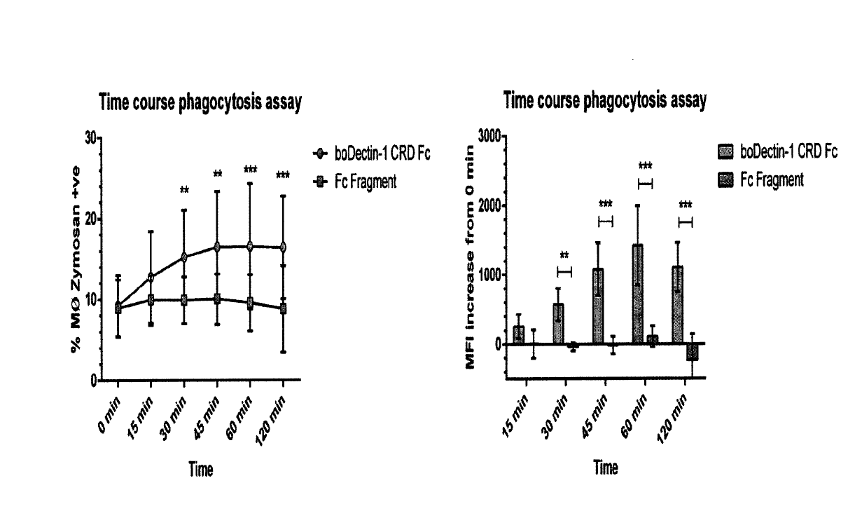

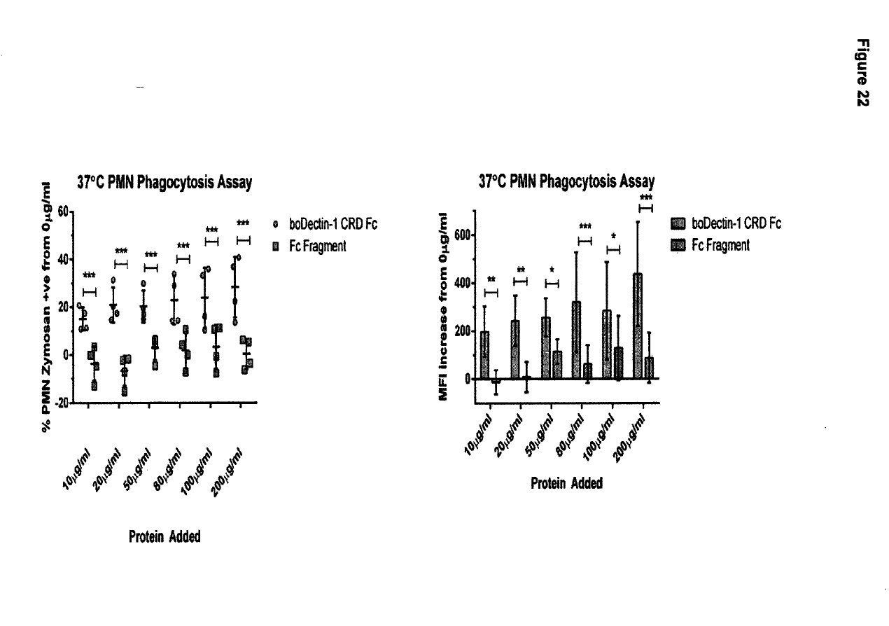

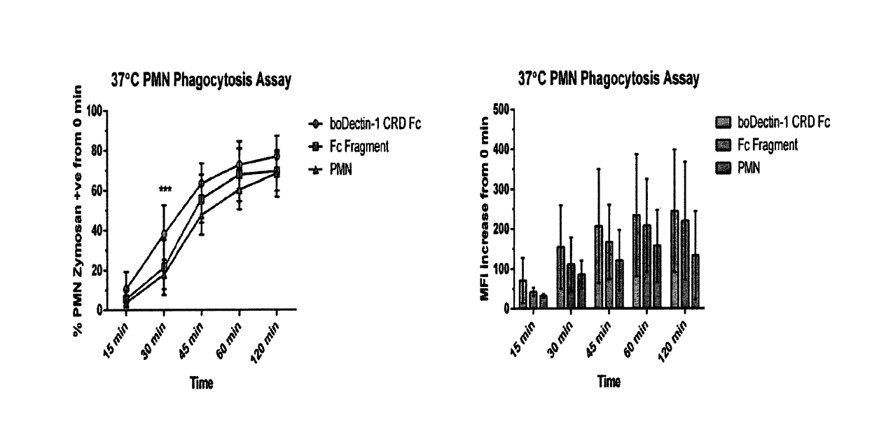

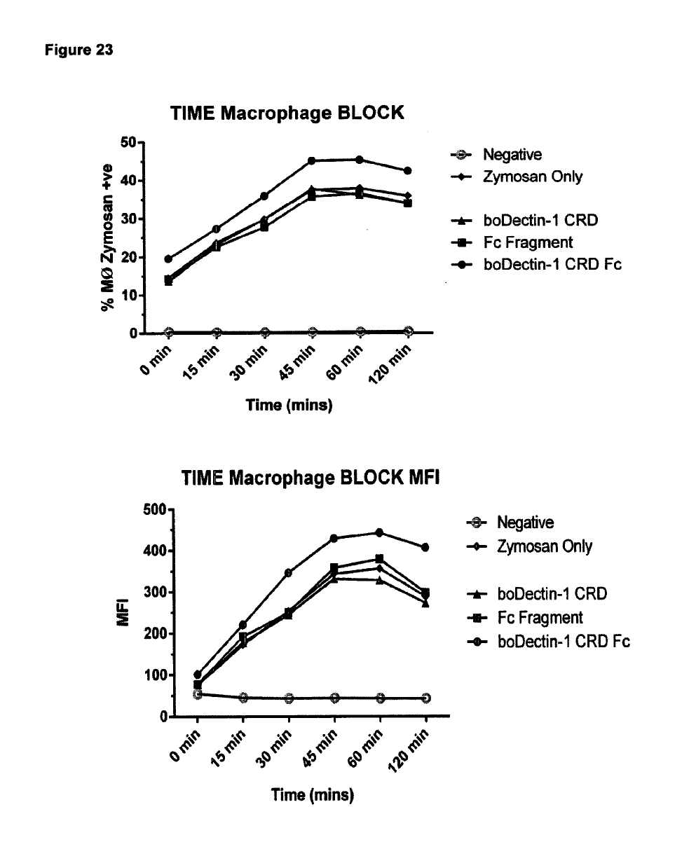

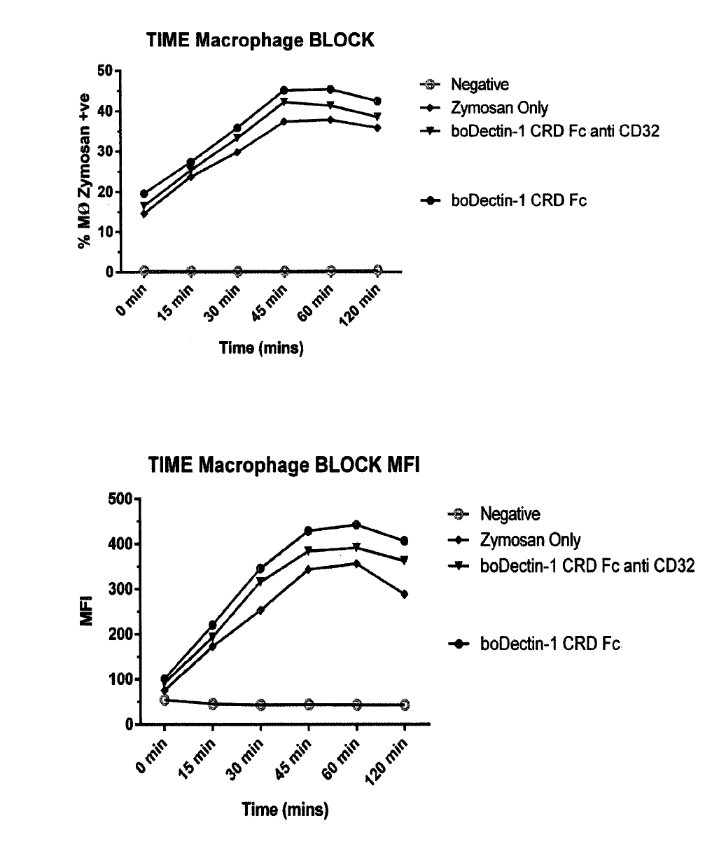

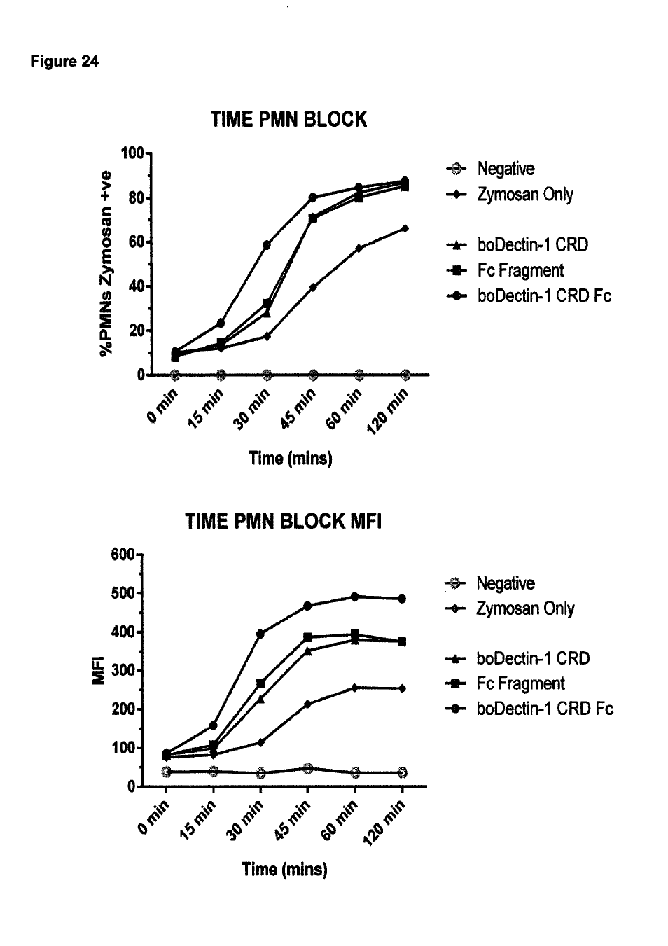

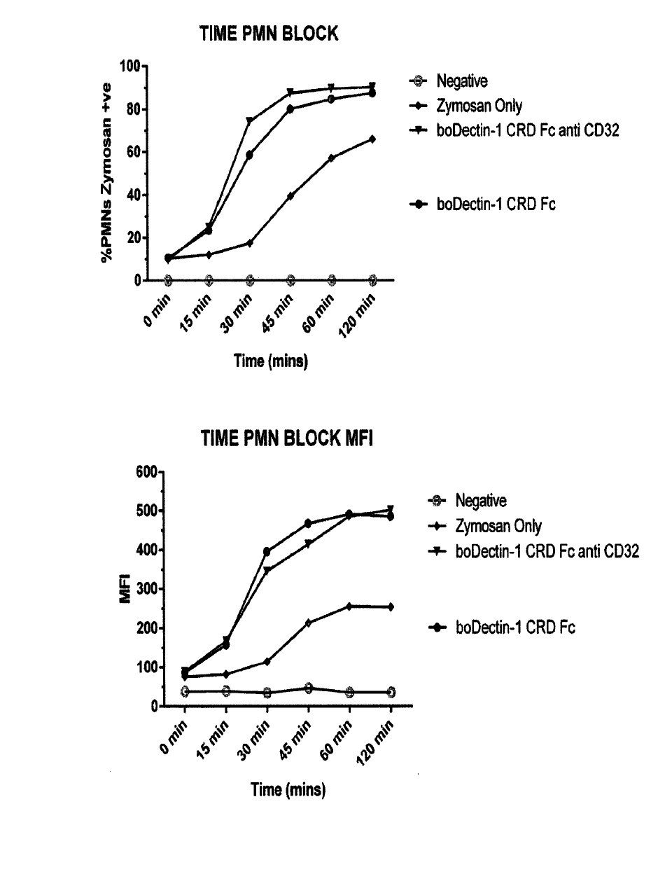

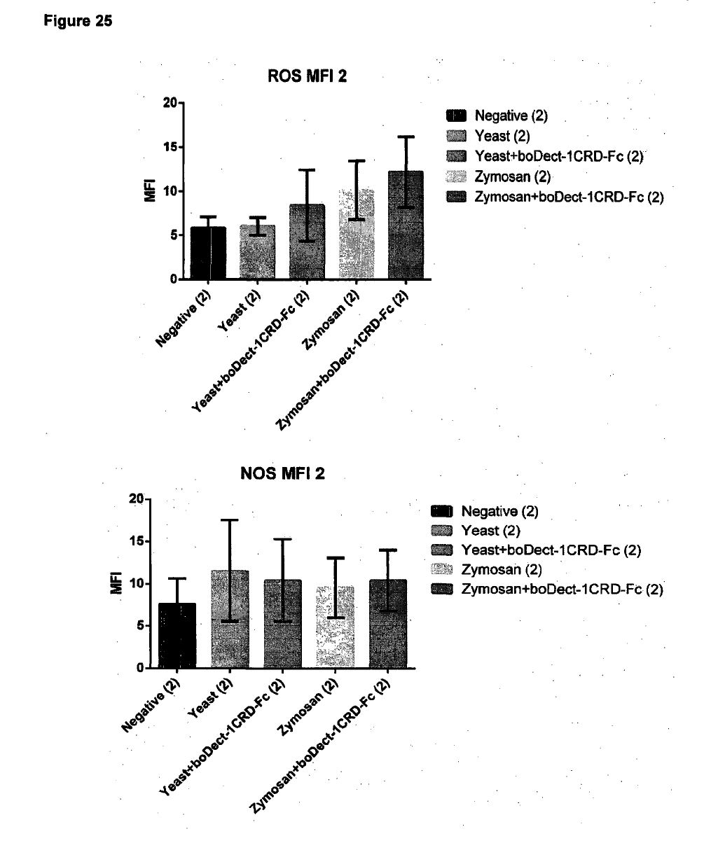

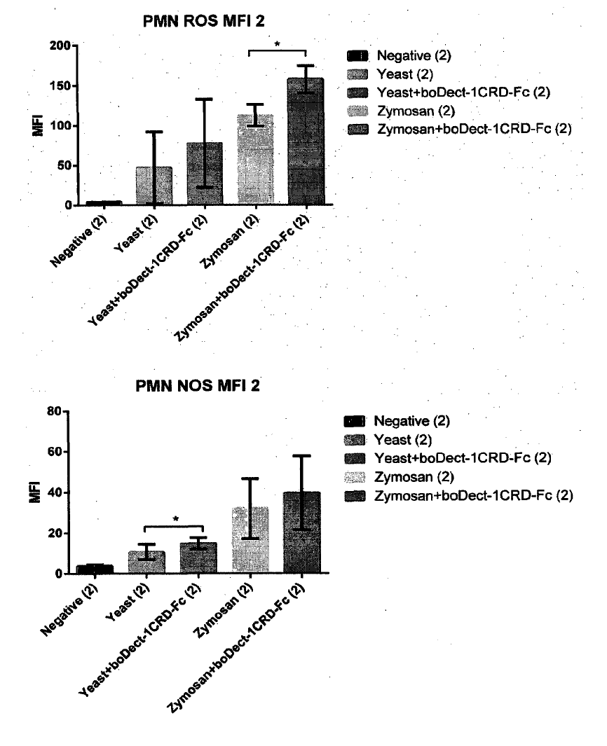

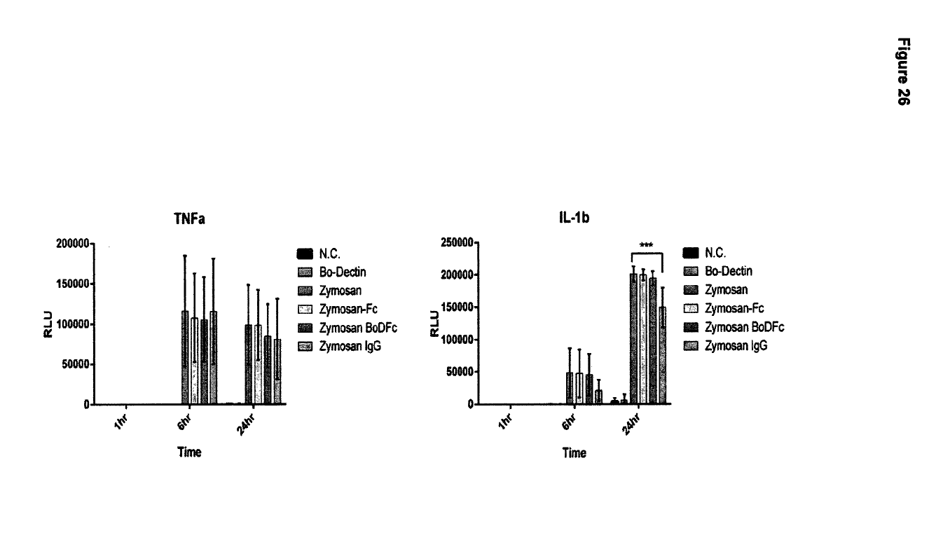

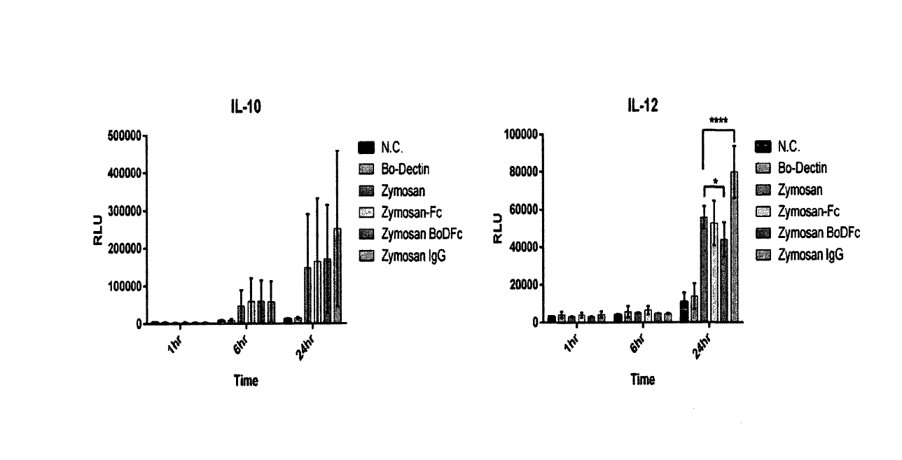

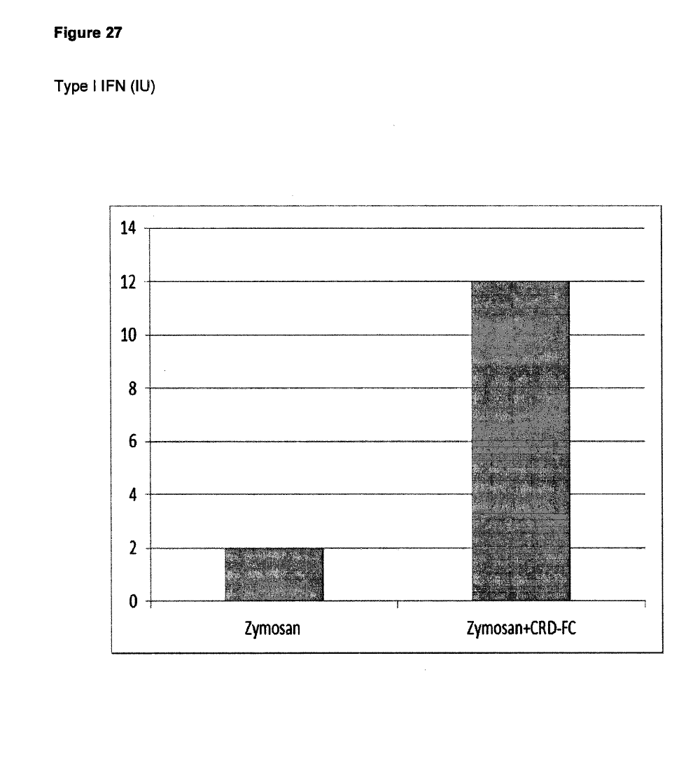

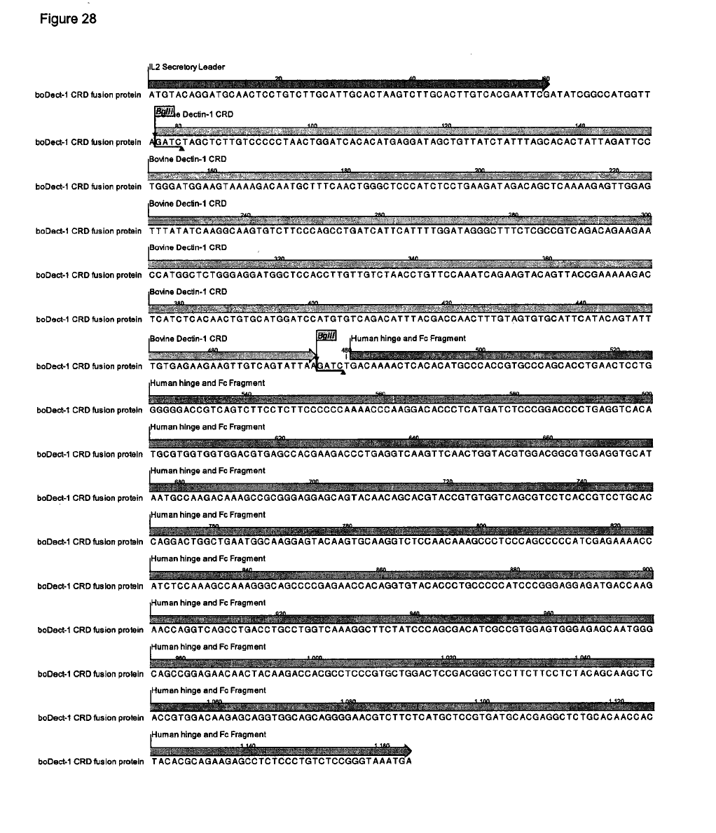

Further assays are described in relation to FIGS. 20 to 26. In conclusion, fusion constructs have been completed and bioactive protein successfully expressed. Such a fusion construct significantly opsonises pathogens in macrophages and neutrophils in a time and concentration dependant manner. Such a fusion construct shows a trend for higher ROS production, which is significant in PMNs. The expression of cytokine IL12 is down regulated in macrophages after the addition of bovine Dectin-1 CRD-Fc.

It is considered that a CRD from a C-type lectin other than, Dectin-1 may be better suited to bacterial killing. For use in cows it may also be more appropriate to use a bovine Fc fragment in place of a human Fc fragment.

### REFERENCES

1. Turner, M. W. and Hamvas, R. M. (2000) Mannose-binding lectin: structure, function, genetics and disease associations. Rev Immunogenet 2, 305-22.
2. Lillie, B. N., Keirstead, N. D., Squires, E. J., Hayes, M. A. (2006) Single-nucleotide polymorphisms in porcine mannan-binding lectin A. Immunogenetics 58, 983-93.
3. Ezekowitz, R. A., Kuhlman, M., Groopman, J. E., Byrn, R. A. (1989) A human serum mannose-binding protein inhibits in vitro infection by the human immunodeficiency virus. The Journal of experimental medicine 169, 185-96.
4. Kelly, P., Jack, D. L., Naeem, A., Mandanda, B., Pollok, R. C., Klein, N. J., Turner, M. W., Farthing, M. J. (2000) Mannose-binding lectin is a component of innate mucosal defense against Cryptosporidium parvum in AIDS. Gastroenterology 119, 1236-42.
5. Neth, O., Jack, D. L., Dodds, A. W., Holzel, H., Klein, N. J., Turner, M. W. (2000) Mannose-binding lectin binds to a range of clinically relevant microorganisms and promotes complement deposition. Infect Immun 68, 688-93.
6. Kuhlman, M., Joiner, K., Ezekowitz, R. A. (1989) The human mannose-binding protein functions as an opsonin. The Journal of experimental medicine 169, 1733-45.
7. Sheriff, S., Chang, C. Y., Ezekowitz, R. A. (1994) Human mannose-binding protein carbohydrate recognition domain trimerizes through a triple alpha-helical coiled-coil. Nat Struct Biol 1, 789-94.
8. Takahashi, M., Mori, S., Shigeta, S., Fujita, T. (2007) Role of MBL-associated serine protease (MASP) on activation of the lectin complement pathway. Adv Exp Med Biol 598, 93-104.
9. Schweinle, J. E., Ezekowitz, R. A., Tenner, A. J., Kuhlman, M., Joiner, K. A. (1989) Human mannose-binding protein activates the alternative complement pathway and enhances serum bactericidal activity on a mannose-rich isolate of Salmonella. J Clin Invest 84, 1821-9.
10. Gaboriaud, C., Teillet, F., Gregory, L. A., Thielens, N. M., Arlaud, G. J. (2007) Assembly of Cl and the MBL- and ficolin-MASP complexes: structural insights. Immunobiology 212, 279-88.
11. Moller-Kristensen, M., Thiel, S., Sjoholm, A., Matsushita, M., Jensenius, J. C. (2007) Cooperation between MASP-1 and MASP-2 in the generation of C3 convertase through the MBL pathway. Int Immunol 19, 141-9.
12. Stuart, L. M., Takahashi, K., Shi, L, Savill, J., Ezekowitz, R. A. (2005) Mannose-binding lectin-deficient mice display defective apoptotic cell clearance but no autoimmune phenotype. J Immunol 174, 3220-6.
13. Casanova, J. L. and Abel, L. (2004) Human Mannose-binding Lectin in Immunity: Friend, Foe, or Both? The Journal of experimental medicine 199, 1295-9.

1. A compound comprising a C-type lectin Carbohydrate Recognition Domain (CRD) and an immunoglobulin Fc domain or a polynucleotide encoding a polypeptide comprising a C-type lectin Carbohydrate Recognition Domain (CRD) and an immunoglobulin Fc domain, for use in treating or preventing a pathogen infection in a non-human, non-murine subject.

2. Use of a compound comprising a C-type lectin Carbohydrate Recognition Domain (CRD) and an immunoglobulin Fc domain, or a polynucleotide encoding a polypeptide comprising a C-type lectin Carbohydrate Recognition Domain (CRD) and an immunoglobulin Fc domain, in the manufacture of a medicament for treating or preventing a pathogen infection in a non-human, non-murine subject.

3. A method for treating or preventing a pathogen infection in a non-human, non-murine subject, the method comprising the step of administering to the subject a compound comprising a C-type lectin Carbohydrate Recognition Domain (CRD) and an immunoglobulin Fc domain, or a polynucleotide encoding a polypeptide comprising a C-type lectin Carbohydrate Recognition Domain (CRD) and an immunoglobulin Fc domain.

4. The method of claim 3 wherein the non-human, non-murine subject is not considered to be an immunocompromised or immunosuppressed subject.

5. The method of claim 3 wherein the non-human, non-murine subject is a ruminant; a livestock; companion or racing animal.

6. The method of claim 3 wherein the pathogen infection comprises infection by a bacterium, optionally Staphylococcus aureus, Streptococcus Uberis, or Streptococcus Agalactiae/dysgalactia .

7. The method of claim 3 wherein the non-human subject is further administered one or more further compounds intended to prevent or aid in resolving the infection, for example an antibiotic or antifungal agent.

8. The compound of claim 1 wherein the CRD and immunoglobulin Fc domain are bovine and the non-human subject is bovine.

9. The compound of claim 1 wherein the CRD is from bovine Mannose binding Lectin (MBL).

10. The compound of claim 1 wherein the immunoglobulin Fc domain is an IgG1 Fc domain.

11. The method of claim 3 wherein the non-human, non-murine subject, optionally bovine subject, has or is at risk of at least one of the group consisting of mastitis, subclinical mastitis, acute mastitis, chronic mastitis, high somatic cell count (high SSC), metritis and endometritis.

12. The method of claim 3 wherein the pathogen infection comprises infection by a mycobacterium, optionally Mycobacterium bovis; or a fungus.

13. The compound of claim 1 wherein the CRD is from bovine DECTIN-1,DC-SIGN or MBL.

14. (canceled)

15. A compound comprising a non-human, non-murine C-type lectin Carbohydrate Recognition Domain (CRD) and a non-human, non-murine immunoglobulin Fc domain, optionally wherein the CRD and the immunoglobulin Fc domain are bovine, optionally wherein the CRD domain is from bovine Mannose Binding Lectin or bovine Dectin-1 or DC-SIGN.

16. A polynucleotide encoding a compound as defined in claim 15.

17. A polynucleotide vector molecule comprising a polynucleotide according to claim 16.

18. A host cell comprising a polynucleotide according to claim 16.

19. (canceled)

20. A pharmaceutical formulation comprising a compound according to claim 15.

21. A pharmaceutical formulation according to claim 20 further comprising an antibiotic or antifungal agent.

22. An antibiotic or antifungal agent for use in treating a non-human, non-murine subject, wherein the subject is administered a compound comprising a C-type lectin Carbohydrate Recognition Domain (CRD) and an immunoglobulin Fc domain.
